(19) United States
(12) Patent Application Publication
(10) Pub. No.: US 2007/0292512 A1
(43) Pub. Date: Dec. 20, 2007

(54) Solid Oral Dosage Form Containing an Enhancer

(75) Inventors: Thomas W. Leonard, Wilmington, NC (US); Edel O'Toole, Marino (IE); Orlagh Feeney, Lisduff (IE)

Correspondence Address: SYNNESTVEDT & LECHNER, LLP, 1101 MARKET STREET, 26TH FLOOR, PHILADELPHIA, PA, 19107-2950, US

(73) Assignee: MERRION RESEARCH II LIMITED, Dublin (IE)

(21) Appl. No.: 11/761,233

(22) Filed: Jun. 11, 2007

Related U.S. Application Data
(60) Provisional application No. 60/812,523, filed on Jun. 9, 2006.

(51) Int. Cl. A61K 38/17; A61K 38/12; A61K 31/19; A61K 31/165; A61K 31/403; A61K 9/24

(52) U.S. Cl. 424/472; 514/12; 514/15; 514/11; 514/616; 514/557; 514/575; 514/356; 514/423; 514/411

(57) ABSTRACT

The invention relates to a pharmaceutical composition, particularly oral dosage forms, comprising a DAC inhibitor in combination with an enhancer to promote absorption of the DAC inhibitor at the GIT cell lining. The enhancer is a medium chain fatty acid or derivative thereof having a carbon chain length of from 6 to 20 carbon atoms. In certain embodiments, the solid oral dosage form is a controlled release dosage form such as a delayed release dosage form.

This application claims the benefit of Provisional Application No. 60/812,523 filed Jun. 9, 2006, which is incorporated herein by reference.

### FIELD OF THE INVENTION

The present invention relates to pharmaceutical compositions and solid oral dosage forms containing an enhancer, and methods of treatment using such compositions. In particular the invention relates to pharmaceutical compositions and solid oral dosage forms comprising a deacetylase (DAC) inhibitor in combination with an enhancer which enhances the bioavailability and/or the absorption of the DAC inhibitor.

### BACKGROUND OF THE INVENTION

The epithelial cells lining the lumenal side of the gastrointestinal tract (GIT) can be a major barrier to drug delivery via oral administration. However, there are four recognized transport pathways which can be exploited to facilitate drug delivery and transport: the transcellular, paracellular, carrier-mediated, and transcytotic transport pathways. The ability of a drug, such as a conventional drug, a peptide, a protein, a macromolecule, or a nano- or microparticulate system, to “interact” with one or more of these transport pathways may result in increased delivery of that drug from the GIT to the underlying circulation.

Certain drugs utilize transport systems for nutrients which are located in the apical cell membranes (i.e., carrier mediated route). Macromolecules may also be transported across the cells in endocytosed vesicles (i.e., transcytosis route). However, many drugs are transported across the intestinal epithelium by passive diffusion either through cells (i.e., transcellular route) or between cells (i.e., paracellular route). Most orally administered drugs are absorbed by passive transport. Drugs which are lipophilic permeate the epithelium by the transcellular route whereas drugs that are hydrophilic are restricted to the paracellular route.

Paracellular pathways occupy less than 0.1% of the total surface area of the intestinal epithelium. Further, tight junctions, which form a continuous belt around the apical part of the cells, restrict permeation between the cells by creating a seal between adjacent cells. Thus, oral absorption of hydrophilic drugs such as peptides can be severely restricted. Other barriers to absorption of drugs may include hydrolyzing enzymes in the lumen brush border or in the intestinal epithelial cells, the existence of the aqueous boundary layer on the surface of the epithelial membrane which may provide an additional diffusion barrier, the mucus layer associated with the aqueous boundary layer and the acid microclimate which creates a proton gradient across the apical membrane. Absorption, and ultimately bioavailability, of a drug may also be reduced by other processes such as P-glycoprotein regulated transport of the drug back into the gut lumen and cytochrome P450 metabolism. The presence of food and/or beverages in the gastrointestinal tract can also interfere with absorption and bioavailability.

Histone acetylation is a reversible modification, with deacetylation being catalyzed by a family of enzymes termed histone deacetylases (HDACs). Grozinger et al., Proc. Natl. Acad. Sci. USA, 96: 4868-4873 (1999), teaches that HDACs are divided into two classes. Grozinger et al. teaches that the human HDAC1, HDAC2, and HDAC3 proteins are members of the first class of HDACs, and discloses new proteins, named HDAC4, HDAC5, and HDAC6, which are members of the second class of HDACs. Kao et al., Genes & Dev., 14: 55-66 (2000), discloses HDAC7, a new member of the second class of HDACs. Van den Wyngaert, FEBS, 478: 77-83 (2000) discloses HDAC8, a new member of the first class of HDACs.

Richon et al., Proc. Natl. Acad. Sci. USA, 95: 3003-3007 (1998), discloses that HDAC activity is inhibited by trichostatin A (TSA), a natural product isolated from Streptomyces hygroscopicus, and by a synthetic compound, suberoylanilide hydroxamic acid (SAHA). Yoshida and Beppu, Exper. Cell Res., 177: 122-131 (1988), teaches that TSA causes arrest of rat fibroblasts at the G₁ and G₂ phases of the cell cycle, implicating HDAC in cell cycle regulation. Indeed, Finnin et al., Nature, 401: 188-193 (1999), teaches that TSA and SAHA inhibit cell growth, induce terminal differentiation, and prevent the formation of tumors in mice. Suzuki et al., U.S. Pat. No. 6,174,905, EP 0847992, JP 258863/96, and Japanese Application No. 10138957, disclose benzamide derivatives that induce cell differentiation and inhibit HDAC activity. Delorme et al., WO 01/38322 and PCT IB01/00683, disclose additional compounds that serve as HDAC inhibitors. Each of the foregoing publications is incorporated herein by reference in their entireties.

The deacetylase inhibitor known as romidepsin (also known as, depsipeptide, FK228, and FR901228), is a cyclic peptide having the structure shown below. 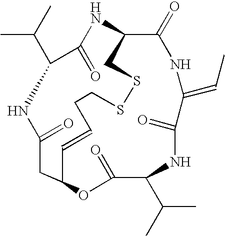
Romidepsin may be produced by a fermentation process utilizing Chromobacterium violaceum as disclosed in U.S. Pat. No. 4,977,138, incorporated herein by reference in its entirety. Following completion of fermentation, romidepsin is recovered and purified by conventional techniques, such as by solvent extraction, chromatography, and/or recrystallization. In addition to isolation of romidepsin from Chromobacterium violaceum, the total synthesis of this compound has now been reported by Kahn et al., J. Am. Chem. Soc. 118:7237-7238 (1996), which is incorporated herein by reference in its entirety. This synthesis involves a 14-step process which provides romidepsin in 18% overall yield. In brief, the synthesis first involved the Carreira catalytic asymmetric aldol reaction to yield a thiol-containing β-hydroxy acid. The peptidic portion of the compound was assembled by standard peptide synthesis methods. The thiol-containing β-hydroxy acid was then coupled to the peptidic portion, and a monocyclic ring generated by formation of the ester (romidepsin) linkage. The bicyclic ring system of romidepsin was then formed upon conversion of the protected thiols to a disulfide linkage.

Romidepsin has been shown to have a potent anti-proliferative effect. For example, romidepsin exhibits in vivo antitumor activity against both human tumor xenografts and murine tumors in mouse models of cancer. Research has shown the inhibition of histone deacetylation to cause cell cycle arrest, differentiation, and apoptotic cell death in cancer cells of various types. Romidepsin is the subject of ongoing study in connection with the treatment of cutaneous T-cell lymphoma, as well as renal cell carcinoma, hormone refractory prostate cancer, breast cancer, and a number of other solid tumors and hematological malignancies including multiple myeloma, chronic lymphocytic leukemia, and acute myeloid leukemia. Romidepsin has also been demonstrated to inhibit the neovascularization in animal models. While not bound by any particular theory as to the mechanism, it is believed that this inhibitory effect is accomplished by suppressing the expression of angiogenic-stimulating factors such as vascular endothelial growth factor or kinase insert domain receptor and by inducing angiogenic-inhibiting factors such as von Hippel Lindau and neurofibromin2. These results indicate that romidepsin may be an anti-angiogenic agent and may contribute to the suppression of tumor expansion, at least in part, by the inhibition of neovascularization. In addition, romidepsin has also been shown to block the hypoxia-stimulated proliferation, invasion, migration, adhesion and tube formation of bovine aortic endothelial cells at the same concentrations at which the agent inhibits HDAC activity of cells.

Romidepsin itself has no apparent chemical structure that appears to interact with the HDAC active-site pocket. Romidepsin, however, is converted by cellular reducing activity to its active, reduced form known as redFK. The disulfide bonds of romidepsin have been shown to be rapidly reduced in cells by cellular reducing activity involving glutathione. In reduced form, redFK possesses two functional sulfhydryl groups at least one of which is believed to be capable of interacting with the zinc in the active-site pocket thereby preventing the access of the substrate.

The inhibitory effect of redFK has been tested against HDAC1 and HDAC2 as class I enzymes and HDAC4 and HDAC6 as class II deacetylases. At low nanomolar concentrations, redFK was shown to be a strong inhibitor of HDAC1 and HDAC2 but relatively weak in inhibiting HDAC4 and HDAC6. More specifically, HDAC6 was shown to be almost insensitive to redFK, romidepsin was 17-23 times weaker than redFK in inhibiting each enzyme, and a dimethyl form of romidepsin showed no inhibitory activity against all of the enzymes.

While redFK has a demonstrated inhibitory activity for class I enzymes, the administration of redFK has been shown to be less active compared to romidepsin in inhibiting in vivo HDAC activity due to rapid inactivation of redFK in medium and serum. As romidepsin is more stable than redFK in both medium and serum, romidepsin can be considered a natural prodrug to inhibit class I enzymes that is activated by reduction to redFK after uptake into the cells. Glutathione-mediated activation also implicates the potential of romidepsin for counteracting glutathione-mediated drug resistance in chemotherapy.

Numerous potential absorption enhancers have been identified. For instance, medium chain glycerides have demonstrated the ability to enhance the absorption of hydrophilic drugs across the intestinal mucosa (see Pharm. Res. (1994), 11, 1148-54). For example, sodium caprate has been reported to enhance intestinal and colonic drug absorption by the paracellular route (see Pharm. Res. (1993) 10, 857-864; Pharm. Res. (1988), 5, 341-346). U.S. Pat. No. 4,656,161 (BASF AG), which is incorporated herein by reference, discloses a process for increasing the enteral absorbability of heparin and heparinoids by adding non-ionic surfactants such as those that can be prepared by reacting ethylene oxide with a fatty acid, a fatty alcohol, an alkylphenol, or a sorbitan or glycerol fatty acid ester.

U.S. Pat. No. 5,229,130 (Cygnus Therapeutics Systems) discloses a composition which increases the permeability of skin to a transdermally administered pharmacologically active agent formulated with one or more vegetable oils as skin permeation enhancers. Dermal penetration is also known to be enhanced by a range of sodium carboxylates (see Int. J. of Pharmaceutics (1994), 108, 141-148). Additionally, the use of essential oils to enhance bioavailability is known (see U.S. Pat. No. 5,665,386 assigned to AvMax Inc.). It is taught that the essential oils act to reduce either, or both, cytochrome P450 metabolism and P-glycoprotein regulated transport of the drug out of the blood stream back into the gut.

Often, however, the enhancement of drug absorption correlates with damage to the intestinal wall. Consequently, limitations to the widespread use of GIT enhancers are frequently determined by their potential toxicities and side effects. Additionally and especially with respect to peptide, protein or macromolecular drugs, the “interaction” of the GIT enhancer with one of the transport pathways should be transient or reversible, such as a transient interaction with or opening of tight junctions so as to enhance transport via the paracellular route.

As mentioned above, numerous potential enhancers are known. However, this has not led to a corresponding number of products incorporating enhancers. One such product currently approved for use in Sweden and Japan is a suppository sold under the trademark Doktacillin® (see Lindmark et al. Pharmaceutical Research (1997), 14, 930-935). The suppository comprises ampicillin and the medium chain fatty acid, sodium caprate (C10).

Provision of a solid oral dosage form which would facilitate the administration of a DAC inhibitor together with an enhancer is desirable. The advantages of solid oral dosage forms over other dosage forms include ease of manufacture, the ability to formulate different controlled release and extended release formulations, and ease of administration. Administration of drugs in solution form does not readily facilitate control of the profile of drug concentration in the bloodstream. Solid oral dosage forms, on the other hand, are versatile and may be modified, for example, to maximize the extent and duration of drug release and to release a drug according to a therapeutically desirable release profile. There may also be advantages relating to convenience of administration including increased patient compliance and to cost of manufacture associated with solid oral dosage forms.

### SUMMARY OF THE INVENTION

According to one aspect of the present invention, the pharmaceutical compositions and dosage forms made therefrom of the present invention comprise a deacetylase (DAC) inhibitor and an enhancer to promote absorption of the DAC inhibitor at the GIT cell lining, wherein the enhancer is a medium chain fatty acid or salt thereof, or a medium chain fatty acid derivative having a carbon chain length of from 6 to 20 carbon atoms; with the provisos that (i) where the enhancer is an ester of a medium chain fatty acid, said chain length of from 6 to 20 carbon atoms relates to the chain length of the carboxylate moiety, and (ii) where the enhancer is an ether of a medium chain fatty acid, at least one alkoxy group has a carbon chain length of from 6 to 20 carbon atoms. The enhancer is thought to work by increasing the absorption of the DAC inhibitor by the gastrointestinal tract, particularly, at the GIT cell lining. In certain embodiments, the enhancer and the resulting compositions and dosage forms are solid at room temperature. In certain embodiments, the pharmaceutical compositions also include at least one auxiliary excipient. In certain embodiments, the DAC inhibitor is an HDAC inhibitor. In certain embodiments, the DAC inhibitor is a TDAC inhibitor. In certain particular embodiments, the DAC inhibitor is romidepsin.

According to another aspect of the present invention, the pharmaceutical compositions and dosage forms made therefrom comprise a DAC inhibitor and an enhancer to promote absorption of the DAC inhibitor at the GIT cell lining, wherein the only enhancer present in the composition is a medium chain fatty acid or salt thereof, or a medium chain fatty acid derivative having a carbon chain length of from 6 to 20 carbon atoms.

The dosage form can be, for example, a tablet, particles (e.g., microparticles, nanoparticles), or a capsule. The multiparticulate forms can be in a tablet or capsule. The tablet can be a single or multilayer tablet having compressed particles in one, a portion, all, or none of the layers. In certain embodiments, the dosage form is a controlled release dosage form. In certain embodiments, the dosage form is a delayed release dosage form. In certain embodiments, the dosage form is an extended release dosage form. The dosage form can be coated (e.g., with a polymer, preferably a rate-controlling or a delayed release polymer). The polymer can also be compressed with the enhancer and drug to form a matrix dosage form such as a controlled, delayed, or extended release matrix dosage form. A coating (e.g., wax, polymer) can be applied to the matrix dosage form.

Other embodiments of the invention include the process of making the dosage forms, and methods for the treatment of a medical condition (e.g., proliferative disease, inflammatory disease, autoimmune disease, cancer) by administering a therapeutically effective amount of a dosage form to a patient.

### BRIEF DESCRIPTION OF THE DRAWINGS

FIG. 1 shows the effect of the sodium salts of C8, C10, C12, C14, C18, and C18:2 with ³H-TRH on TEER (Ωcm²) in Caco-2 monolayers at time 0 and at 30 min. intervals up to 2 hours as described in Example 1.

FIG. 2 shows the effect of the sodium salts of C8, C10, C12, C14, C18, and C18:2 on Pₐₚₚ for ³H-TRH transport in Caco-2 monolayers as described in Example 1.

FIG. 3 shows the serum TRH concentration-time profiles following interduodenal bolus dose of 500 μg TRH with NaC8 or NaC10 (35 mg) enhancer present according to the closed loop rat model described in Example 1.

FIG. 4 shows the serum TRH concentration-time profiles following interduodenal bolus dose of 1000 μg TRH with NaC8 or NaC10 (35 mg) enhancer present according to the closed loop rat model described in Example 1.

FIG. 5 shows the APTT response over a period of 4 hours following administration of USP heparin (1000 IU) with different sodium caprate (C10) levels (10 and 35 mg) according to the closed loop rat model described in Example 2.

FIG. 6 shows the anti-factor Xₐ response over a period of 5 hours following administration of USP heparin (1000 IU) in the presence of different sodium caprylate (C8) levels (10 mg and 35 mg) according to the closed loop rat model described in Example 2.

FIG. 7 shows the anti-factor Xₐ response over a period of five hours following administration of USP heparin (1000 IU) in the presence of different sodium caprate (C10) levels (10 mg and 35 mg) according to the closed loop rat model described in Example 2.

FIG. 8 shows the mean anti-factor Xₐ response in dogs over a period of time up to 8 hours following administration of: a) s.c. USP heparin solution (5000 IU); b) oral uncoated instant release tablet formulation containing USP heparin (90000 IU) and NaC10; c) oral uncoated instant release tablet formulation containing USP heparin (90000 IU) and NaC8; and d) oral uncoated sustained release tablet formulation containing USP heparin (90000 IU) and sodium caprate prepared according to the invention as described in Example 2.

FIG. 9 shows the anti-factor Xₐ response over a period of three hours following intraduodenal administration to rats of phosphate buffered saline solutions of parnaparin sodium (low molecular weight heparin (LMWH)) (1000 IU), in the presence of 35 mg of different enhancers such as sodium caprylate (C8), sodium nonanoate (C9), sodium caprate (C10), sodium undecanoate (C11), sodium laurate (C12), and different 50:50 binary mixtures of enhancers, to rats (n=8) in an open loop model. The reference product comprised administering 250 IU parnaparin sodium subcutaneously. The control solution comprised administering a solution containing 1000 IU parnaparin sodium without any enhancer intraduodenally.

FIG. 10 shows the mean plasma levels of leuprolide over a period of eight hours following intraduodenal administration of solutions of leuprolide (20 mg) containing different levels of sodium caprate (0.0 g (control), 0.55 g, 1.1 g) to dogs.

FIG. 11 shows the mean anti-factor Xₐ response in dogs over a period of eight hours following oral administration of parnaparin sodium (90,000 IU) in the presence of 550 mg sodium caprate, as both a solution (10 ml) and an instant release tablet dosage form.

FIG. 12 shows the mean anti-factor Xₐ response in humans over a period of 24 hours following oral administration of parnaparin sodium (90,000 IU) in the presence of sodium caprate, as both a solution (240 ml) and as an instant release tablet dosage form

FIG. 13 shows the mean anti-factor Xₐ response in humans over a period of 24 hours following intrajejunal administration of 15 ml solutions containing different doses of parnaparin sodium (20,000 IU, 45,000 IU, 90,000 IU) in the presence of different doses of sodium caprate (0.55 g, 1.1 g, 1.65 g)

FIG. 14 shows the mean anti-factor Xₐ response in dogs over a period of 8 hours following oral administration of 45,000 IU parnaparin sodium as: (a) instant release capsules containing 0.55 g sodium caprate, (b) Eudragit L coated rapidly disintegrating tablets containing 0.55 g sodium caprate, and (c) Eudragit L coated rapidly disintegrating tablets without enhancer.

FIG. 15 shows the mean anti-factor Xₐ response in dogs over a period of 8 hours following co-administration of 45,000 IU LMWH and 0.55 g sodium caprate orally, intrajejunally, and intracolonically compared to subcutaneous administration.

FIG. 16 shows group mean data for intraduodenal administration of different formulations of romidepsin and an enhancer.

### DETAILED DESCRIPTION OF THE INVENTION

As used in this specification and appended claims, the singular forms “a”, “an” and “the” include plural referents unless the content clearly dictates otherwise. Thus, for example, reference to “an enhancer” includes a mixture of two or more enhancers, reference to “a DAC inhibitor” includes a mixture of two or more DAC inhibitors, and reference to “an additional drug” includes a mixture of two or more additional drugs, the like.

As used herein, the terms “deacetylase” and “DAC” are intended to refer to any deactylase activity in the cell. In certain embodiments, the deacetylase activity is histone deacetylase (HDAC) activity. In certain embodiments, the deacetylase activity is tubulin deacetylase (TDAC) activity. In certain embodiments, deacetylase activity refers to the deacetylation of other proteins or biological molecules in the cell. In certain embodiments, the deacetylase activity removes the acetyl group from the ε-amino group of a lysine residue of a protein or peptide.

As used herein, the terms “histone deacetylase” and “HDAC” are intended to refer to any one of a family of enzymes that remove acetyl groups from the ε-amino groups of lysine residues of a histone. Histone deacetylases are thought to play an important role in cellular proliferation. Unless otherwise indicated by context, the term “histone” is meant to refer to any histone protein, including H1, H2A, H₂B, H3, H4, and H5, from any species. Histone deacetylases may include class I and class II enzymes, and may also be of human origin, including, but not limited to, HDAC-1, HDAC-2, HDAC-3, HDAC4, HDAC-5, HDAC-6, HDAC-7, HDAC-8, HDAC-9, HDAC-10, and HDAC-11. In certain embodiments, the histone deacetylase is derived from a mammalian source (e.g. rat, mouse, rabbit, dog, cat, pig, primate, human, etc.). In certain particular embodiments, the histone deacetylase is derived from a human source. In some embodiments, the histone deacetylase is derived from a protozoal, bacterial, or fungal source.

As used herein, the terms “deacetylase inhibitor,” “DAC inhibitor” and “drug” are intended to refer to a compound which is capable of interacting with a deacetylase enzyme and inhibiting its enzymatic activity. The phrase “inhibiting deacetylase enzymatic activity” means reducing the ability of a deacetylase to remove an acetyl group from a substrate. In certain embodiments, the substrate is an acetylated ε-amino group of a lysine residue. In some embodiments, such reduction of deacetylase activity is at least about 5%, at least about 10%, at least about 20%, at least about 25%, at least about 30%, at least about 40%, at least about 50%, at least about 60%, at least about 70%, at least about 75%, at least about 80%, or at least about 90%. In other embodiments, deacetylase activity is reduced by at least 95% or at least 99%. Suitable DAC inhibitors include, for example, short-chain fatty acids such as butyrate, phenylbutyrate, pivaloyloxymethyl butyrate, N-hydroxy-4-(3-methyl-2-phenyl-butyrylamino)-benzamide, 4-(2,2-Dimethyl-4-phenylbutyrylamino)-N-hydroxybenzamide, valproate and valproic acid; hydroxamic acids and their derivatives such as suberoylanilide hydroxamic acid (SAHA) and its derivatives, oxamflatin, M-carboxycinnamic acid bishydroxamide, 6-(3-benzoyl-ureido)-hexanoic acid hydroxyamide, suberic bishydroxamate (SBHA), N-hydroxy-7-(2-naphthylthio) heptanomide (HNHA), nicotinamide, scriptaid (SB-556629), scriptade, splitomicin, lunacin, ITF2357, A-161906, NVP-LAQ824, LBH589, pyroxamide, CBHA, 3-Cl-UCHA, SB-623, SB-624, SB-639, SK-7041; propenamides such as 3-(4-dimethylaminophenyl)-N-hydroxy-2-propenamide, 2-amino-8-oxo-9,10-epoxy-decanoyl, 3-(4-aroyl-1H-pyrrol-2-yl)-N-hydroxy-2-propenamide, and MC 1293; aroyl pyrrolyl hydroxyamides such as APHA Compound 8; trichostatins such as trichostatin A and trichostatin C; cyclic tetrapeptides such as trapoxin including trapoxin A and trapoxin B, romidepsin, antanapeptins A-D, HC-toxin, chlamydocin, diheteropeptin, WF-3161, Cyl-1, Cyl-2, apicidin, FR225497, FR901375, spiruchostatins such as spiruchostatin A, spiruchostatin B and spiruchostatin C, salinamides such as salinamide A and salinamide B, and cyclic-hydroxamic-acid-containing peptides (CHAPs); benzamides such as M344, MS-275, CI-994 (N-acetyldinaline), tacedinaline and sirtinol; tricyclic lactam and sultam derivatives; acetate derivatives of amijiol, organosulfur compounds such as diallyl disulfide and sulforaphane; electrophilic ketones such as α-ketoamide and trifluoromethylketone; pimeloylanilide o-aminoanilide (PAOA); depudecin; psammaplins such as psammaplin A and psammaplin F; tubacin; curcumin; histacin; 6-Chloro-2,3,4,9-tetrahydro-1H-carbazole-1-carboxamide, CRA-024781; CRA-026440; CG1521; PXD101; G2M-777, CAY10398, CTPB, MGCD0103, and BL1521. The term “DAC inhibitor” also includes all analogs, isomers, derivatives, salts, enantiomers, diastereomers, stereoisomers, tautomers, and other forms thereof including optically pure enantiomers or steroeisomers, mixtures, racemates, as well as all pharmaceutically acceptable derivatives thereof. In one embodiment, the DAC inhibitor is romidepsin.

As used herein, the term “romidepsin” refers to a natural product of the chemical structure: 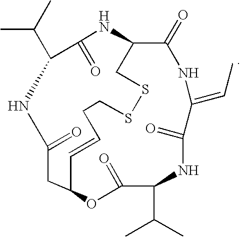
Romidepsin is a potent HDAC inhibitor and is also known in the art by the names FK228, FR901228, NSC630176, or depsipeptide. The identification and preparation of romidepsin is described in U.S. Pat. No. 4,977,138, which is incorporated herein by reference. The molecular formula is C₂₄H₃₆N₄O₆S₂; and the molecular weight is 540.71. Romidepsin has the chemical name, (1S,4S,10S,16E,21R)-7-[(2Z)-ethylidene]-4,2,1-diisopropyl-2-oxa-12,13-dithia-5,8,20,23-tetraazabicyclo[8.7.6]tricos-16-ene-3,6,9,19,22-pentanone. Romidepsin has been assigned the CAS number 128517-07-7. In crystalline form, romidepsin is typically a white to pale yellowish white crystal or crystalline powder. The term “romidepsin” encompasses this compound and any pharmaceutically acceptable salt forms thereof. In certain embodiments, the term “romidepsin” may also include pro-drugs, esters, protected forms, and derivatives thereof.

The drug may be provided in any suitable phase state including as a solid, liquid, solution, suspension, and the like. When provided in solid particulate form, the particles may be of any suitable size or morphology and may assume one or more crystalline, semi-crystalline, and/or amorphous forms. The drug can be included in nano- or microparticulate drug delivery systems in which the drug is, or is entrapped within, encapsulated by, attached to, or otherwise associated with, a nano- or microparticle.

As used herein, a “therapeutically effective amount of a DAC inhibitor” refers to an amount of DAC inhibitor that elicits a therapeutically useful response in an animal, preferably a mammal, most preferably a human. In certain embodiments, the amount is sufficient to inhibit the proliferation of unwanted cells (e.g., cancerous cells, inflammatory cells, undesired cells).

As used herein, the term “enhancer” refers to a compound or mixture of compounds which is capable of enhancing the transport of a drug across the GIT in an animal such as a human. In certain embodiments, the enhancer is a medium chain fatty acid, or salt thereof, or a medium chain fatty acid derivative, or salt thereof, having a carbon chain length of from 6 to 20 carbon atoms; with the provisos that (i) where the enhancer is an ester of a medium chain fatty acid, said chain length of from 6 to 20 carbon atoms relates to the chain length of the carboxylate moiety, and (ii) where the enhancer is an ether of a medium chain fatty acid, at least one alkoxy group has a carbon chain length of from 6 to 20 carbon atoms. In certain embodiments, the enhancer is a sodium salt of a medium chain fatty acid. Other salts of medium chain fatty acids may also be used including ammonium, lithium, potassium, magnesium, aluminum, and calcium salts. In certain particular embodiments, the enhancer is sodium caprate. In certain embodiments, the enhancer is a solid at room temperature.

As used herein, the term “medium chain fatty acid derivative” includes fatty acid salts, esters, ethers, acid halides, carbamates, carbonates, amines, ureas, amides, anhydrides, carboxylate esters, nitrites, as well as glycerides such as mono-, di-, or tri-glycerides. The carbon chain may be characterized by various degrees of saturation or unsaturation. In other words, the carbon chain may be, for example, fully saturated or partially unsaturated (i.e., containing one or more carbon-carbon double or triple bonds). The term “medium chain fatty acid derivative” is also meant to encompass medium chain fatty acids wherein the end of the carbon chain opposite the acid group (or derivative) is functionalized with one of the above mentioned moieties (e.g., an ester, ether, acid halide, hydroxyl, carbamate, carbonate, amine, urea, amide, anhydride, carboxylate ester, nitrile, or glyceride moiety). Such difunctional fatty acid derivatives thus include for example diacids and diesters (the functional moieties being of the same kind) and also difunctional compounds comprising different functional moieties, such as amino acids and amino acid derivatives, for example, a medium chain fatty acid or an ester or a salt thereof comprising an amide moiety at the opposite end of the fatty acid carbon chain to the acid or ester or salt thereof. Exemplary salts include alkali and alkaline earth metal salts such as lithium, sodium, potassium, calcium, magnesium, aluminum, etc. The salts may also be organic salts such as ammonium salts.

As used herein, a “therapeutically effective amount of an enhancer” refers to an amount of enhancer that allows for uptake of a therapeutically effective amount of an orally administered drug (e.g., a DAC inhibitor such romidepsin). It has been shown that the effectiveness of an enhancer in enhancing the gastrointestinal delivery of poorly permeable drugs is dependent on the site of administration (see Examples 6, 7 and 12).

The enhancer of the present invention interacts in a transient and reversible manner with the GIT cell lining increasing permeability and facilitating the absorption of otherwise poorly permeable molecules. In certain embodiments, enhancers include (i) medium chain fatty acids and their salts, (ii) medium chain fatty acid esters of glycerol and propylene glycol, and (iii) bile salts. In one embodiment, the enhancer is a medium chain fatty acid salt, ester, ether, amide, or other derivative of a medium chain fatty acid which is, preferably, solid at room temperature and which has a carbon chain length of from 8 to 14 carbon atoms; with the provisos that (i) where the enhancer is an ester of a medium chain fatty acid, said chain length of from 8 to 14 carbon atoms relates to the chain length of the carboxylate moiety, and (ii) where the enhancer is an ether of a medium chain fatty acid, at least one alkoxy group has a carbon chain length of from 8 to 14 carbon atoms. In certain embodiments, the chain length is an even number of carbon atoms (e.g., 8, 10, 12, 14). In other embodiments, the chain length is an odd number of carbon atoms (e.g., 9, 11, 13, 15). In certain embodiments, the carbon chain length is 8. In other embodiments, the carbon chain length is 10. In still other embodiments, the carbon chain length is 12. In certain embodiments, the enhancer is caprylic acid or a salt form thereof. In certain embodiments, the enhancer is capric acid of a salt form thereof. In certain embodiments, the enhancer is lauric acid or a salt thereof. In certain particular embodiments, the enhancer is a sodium salt of a medium chain fatty acid, the medium chain fatty acid having a carbon chain length of from 8 to 14 carbon atoms; the sodium salt being solid at room temperature. In a further embodiment, the enhancer is sodium caprylate, sodium caprate, or sodium laurate. The drug and enhancer can be present in a ratio of from 1:100,000 to 100:1 (drug:enhancer). In certain embodiments, the ratio of drug to enhancer ranges from 1:10000 to 10:1. In certain embodiments, the ratio of drug to enhancer ranges from 1:5000 to 10:1. In certain embodiments, the ratio of drug to enhancer ranges from 1:1000 to 10:1. In certain embodiments, the ratio of drug to enhancer ranges from 1:1000 to 1:1. In certain embodiments, the ratio of drug to enhancer ranges from 1:500 to 1:1. In certain embodiments, the ratio of drug to enhancer ranges from 1:100 to 1:1. In certain embodiments, the ratio of drug to enhancer ranges from 1:10 to 10:1. In certain embodiments, the ratio of drug to enhancer ranges from 1:1 to 10:1. In certain embodiments, the ratio of drug to enhancer ranges from 1:1 to 100:1.

As used herein, the term “rate controlling polymer material” includes hydrophilic polymers, hydrophobic polymers, and mixtures of hydrophilic and/or hydrophobic polymers that are capable of controlling the release of the drug from a solid oral dosage form of the present invention. The polymer may be a synthetic or natural polymer. Suitable rate controlling polymer materials include those selected from the group consisting of hydroxyalkyl celluloses such as hydroxypropyl cellulose, hydroxypropylmethyl cellulose, hydroxypropylmethyl cellulose phthalate, and hydroxypropylmethyl cellulose acetate succinate; poly(ethylene) oxide; alkyl celluloses such as ethyl cellulose and methyl cellulose; carboxymethyl cellulose; hydrophilic cellulose derivatives; polyethylene glycol; polyvinylpyrrolidone; cellulose acetates such as cellulose acetate butyrate, cellulose acetate phthalate, and cellulose acetate trimellitate; polyvinyl acetates such as polyvinyl acetate; polyvinyl acetate phthalate, and polyvinyl acetaldiethylamino acetate; polyacrylates, polyesters, polyanhydrides, and polyalkylmethacrylates. Other suitable hydrophobic polymers include polymers and/or copolymers derived from acrylic or methacrylic acid and their respective esters, zein, waxes, shellac and hydrogenated vegetable oils.

Rate controlling polymer materials that are particularly useful in the practice of the present invention are polyacrylic acid, polyacrylate, polymethacrylic acid and polymethacrylate polymers such as those sold under the Eudragit® trade name (Rohm GmbH, Darmstadt, Germany) specifically Eudragit® L, Eudragit® S, Eudragit® RL, Eudragit® RS, Eudragit L100-55 and Acryl-Eze® MP (Colorcon, West Point, Pa.) coating materials and mixtures thereof. Some of these polymers can be used as delayed release polymers to control the site where the drug is released. They include polymethacrylate polymers such as those sold under the Eudragit® trade name, specifically Eudragit® L, Eudragit® S, Eudragit RL, Eudragit® RS, Eudragit® L100-55, and Acryl-Eze® MP coating materials and mixtures thereof.

A solid oral dosage form according to the present invention may be a tablet, particles (e.g., microparticles, nanoparticles), or a capsule. A preferred solid oral dosage form is a delayed release dosage form which minimizes the release of the drug and enhancer in the stomach, and hence the dilution of the local enhancer concentration therein, and releases the drug and enhancer in the intestine. A particularly preferred solid oral dosage form is a delayed release rapid onset dosage form. Such a dosage form minimizes the release of the drug and enhancer in the stomach, and hence the dilution of the local enhancer concentration therein, but releases the drug and enhancer rapidly once the appropriate site in the intestine has been reached, maximizing the delivery of the drug by maximizing the local concentration of drug and enhancer at the site of absorption. The drug and enhancer are typically present at the same site for absorption. In certain embodiments, the increase the solubility of the drug and/or enhancer at the desired site in the intestines a solubilizer is used.

As used herein, the term “tablet” includes, but is not limited to, immediate release (IR) tablets, sustained release (SR) tablets, matrix tablets, multilayer tablets, multilayer matrix tablets, extended release tablets, delayed release tablets, and pulsed release tablets, any or all of which may optionally be coated with one or more coating materials, including polymeric or wax coating materials, such as enteric coatings, rate-controlling coatings, semi-permeable coatings, and the like. The term “tablet” also includes osmotic delivery systems in which a DAC inhibitor is combined with an osmagent (and optionally other excipients) and coated with a semi-permeable membrane, the semi-permeable membrane defining an orifice through which the drug compound may be released. Tablet solid oral dosage forms particularly useful in the practice of the invention include those selected from the group consisting of IR tablets, SR tablets, coated IR tablets, matrix tablets, coated matrix tablets, multilayer tablets, coated multilayer tablets, multilayer matrix tablets and coated multilayer matrix tablets. In certain embodiments, the tablet dosage form is an enteric coated tablet dosage form. In certain embodiments, the tablet dosage form is an enteric coated rapid onset tablet dosage form.

As used herein, the term “capsule” includes instant release capsules, sustained release capsules, coated instant release capsules, coated sustained release capsules, delayed release capsules, and coated delayed release capsules. In one embodiment, the capsule dosage form is an enteric coated capsule dosage form. In another embodiment, the capsule dosage form is an enteric coated rapid onset capsule dosage form.

The terms “particles” or “multiparticulate” as used herein refers to a plurality of discrete particles, granules, pellets, or mini-tablets, regardless of size or morphology, and mixtures or combinations thereof. If the oral form is a multiparticulate capsule, hard or soft gelatin capsules can suitably be used to contain the multiparticulate material. Alternatively a sachet can suitably be used to contain the multiparticulate material. The multiparticulate material may be coated with a layer containing rate controlling polymer material. The multiparticulate oral dosage form may comprise a blend of two or more populations of particles, granules, pellets, or mini-tablets having different agents to be delivered. For example, one population of particles may include the enhancer, and another population of particles may include the drug (e.g., romidepsin). The multiparticulate oral dosage form may also comprise a blend of two or more populations of particles, granules, pellets, or mini-tablets having different in vitro and/or in vivo release characteristics. For example, a multiparticulate oral dosage form may comprise a blend of an instant release component and a delayed release component contained in a suitable capsule. In one embodiment, the multiparticulate dosage form comprises a capsule containing delayed release rapid onset minitablets. In another embodiment, the multiparticulate dosage form comprises a delayed release capsule comprising instant release minitablets. In a further embodiment, the multiparticulate dosage form comprises a capsule comprising delayed release granules. In yet another embodiment, the multiparticulate dosage form comprises a delayed release capsule comprising instant release granules.

In another embodiment, the multiparticulate together with one or more auxiliary excipient materials may be compressed into tablet form such as a single layer or multilayer tablet. Typically, a multilayer tablet may comprise two layers containing the same or different levels of the same active ingredient having the same or different release characteristics. Alternatively, a multilayer tablet may contain a different active ingredient(s) in each layer. Such a tablet, either single layered or multilayered, can optionally be coated with a controlled release polymer so as to provide additional controlled release properties.

A number of embodiments of the invention will now be described. In each case the DAC inhibitor may is present in any amount which is sufficient to elicit a therapeutic effect. As will be appreciated by those skilled in the art, the actual amount of DAC inhibitor used will depend on, among other things, the potency of the DAC inhibitor that is used, the specifics of the patient and the therapeutic purpose for which the DAC inhibitor is being used. In embodiments in which romidepsin is the DAC inhibitor, the amount of romidepsin used may be in the range of from about 0.5 mg/m² to about 300 mg/m², and may be administered in amounts suitable to achieve blood plasma concentrations of from about 1 ng/mL to about 500 ng/mL. In certain embodiments, the amount of romidepsin used is in the range of from about 0.5 mg/m² to about 10 mg/m². In certain embodiments, the amount of romidepsin used is in the range of from about 1 mg/m² to about 25 mg/m². In certain embodiments, the amount of romidepsin used is in the range of from about 10 mg/m² to about 50 mg/m². In certain embodiments, the amount of romidepsin used is in the range of from about 25 mg/m² to about 200 mg/m². In certain embodiments, the amount of romidepsin used is in the range of from about 25 mg/m² to about 75 mg/m². In certain embodiments, the amount of romidepsin used is in the range of from about 25 mg/m² to about 100 mg/m². In certain embodiments, the amount of romidepsin used is in the range of from about 50 mg/m² to about 150 mg/m². In certain embodiments, the amount of romidepsin used is in the range of from about 100 mg/m² to about 200 mg/m². In certain embodiments, the amount of romidepsin used is in the range of from about 200 mg/m² to about 300 mg/m². In certain embodiments, the amount of romidepsin used is greater than 300 mg/m². The enhancer is suitably present in any amount sufficient to allow for uptake of therapeutically effective amounts of the drug via oral administration. In one embodiment, the drug and the enhancer are present in a ratio of from 1:100,000 to 100:1 (drug:enhancer). In certain embodiments, the ratio of drug to enhancer ranges from 1:10000 to 10:1. In certain embodiments, the ratio of drug to enhancer ranges from 1:5000 to 10:1. In certain embodiments, the ratio of drug to enhancer ranges from 1:1000 to 10:1. In certain embodiments, the ratio of drug to enhancer ranges from 1:1000 to 1:1. In certain embodiments, the ratio of drug to enhancer ranges from 1:500 to 1:1. In certain embodiments, the ratio of drug to enhancer ranges from 1:100 to 1:1. In certain embodiments, the ratio of drug to enhancer ranges from 1:10 to 10:1. In certain embodiments, the ratio of drug to enhancer ranges from 1:1 to 10:1. In certain embodiments, the ratio of drug to enhancer ranges from 50:1 to 100:1. In certain embodiments, the ratio of drug to enhancer ranges from 1:1 to 100:1. The actual ratio of drug to enhancer used will depend on, among other things, the potency of the particular drug and/or the enhancing activity of the particular enhancer.

In one embodiment, there is provided a pharmaceutical composition and a solid oral dosage form made therefrom comprising a DAC inhibitor and, as an enhancer to promote absorption of the DAC inhibitor at the GIT cell lining, a medium chain fatty acid, or salt form thereof, or a medium chain fatty acid derivative, or salt form thereof, having a carbon chain length of from 6 to 20 carbon atoms. In certain embodiments, the enhancer and/or the composition are solids at room temperature. In one such embodiment, the HDAC inhibitor is romidepsin.

In another embodiment, there is provided a pharmaceutical composition and an oral dosage form made therefrom, comprising a DAC inhibitor and, as an enhancer to promote absorption of the HDAC inhibitor at the GIT cell lining, wherein the only enhancer present in the composition is a medium chain fatty acid, or salt form thereof, or a medium chain fatty acid derivative, or salt form thereof, having a carbon chain length of from 6 to 20 carbon atoms. In one such embodiment, the DAC inhibitor is romidepsin. In certain embodiments, the composition includes romidepsin as the DAC inhibitor and sodium caprylate as the enhancer. In certain embodiments, the compositions include romidepsin as the DAC inhibitor and sodium caprate as the enhancer. In certain embodiments, the composition includes romidepsin and sodium laurate. Any of these compositions may include other pharmaceutically acceptable excipients such as filler, agents to control release kinetics, wetting agents, etc. In certain embodiments, the excipient is polyvinylpyrrolidone.

In a further embodiment, there is provided a multilayer tablet comprising a composition of the present invention. Typically such a multilayer tablet comprises a first layer containing a drug (e.g., romidepsin) and an enhancer in an instant release form and at least a second layer containing a drug (e.g., romidepsin) and an enhancer in a modified release form. As used herein, the term “modified release” includes sustained, delayed, or otherwise controlled release of a drug upon administration to a patient. In an alternative embodiment, a multilayer tablet may comprise a first layer containing a drug and at least a second layer containing an enhancer. The drug in the first and the at least second layer may be the same or different, and each layer may independently comprise further excipients chosen to modify the release of the drug and/or the enhancer. Thus the drug and the enhancer may be released from the respective first and at least second layers at rates which are the same or different. Alternatively, each layer of the multilayer tablet may comprise both drug and enhancer in the same or different amounts. In one such multilayer tablet embodiment, the drug is a DAC inhibitor is romidepsin. Other drugs included in the tablet may be cytotoxic agents or anti-proliferative agents. In certain other embodiments, the other drug is an anti-inflammatory agent.

In yet another embodiment, the present invention provides a multiparticulate composition comprising a HAC inhibitor (e.g., romidepsin) and an enhancer. The multiparticulate composition may comprise particles, granules, pellets, mini-tablets, or combinations thereof, and the drug and the enhancer may be contained in the same or different populations of particles, granules, pellets, or mini-tablets making up the multiparticulate composition. In multiparticulate embodiments, sachets and capsules such as hard or soft gelatin capsules can suitably be used to contain the multiparticulate material. A multiparticulate dosage form may comprise a blend of two or more populations of particles, granules, pellets, or mini-tablets having different in vitro and/or in vivo release characteristics. For example, a multiparticulate dosage form may comprise a blend of an immediate release component and a delayed release component contained in a suitable capsule. In one such multiparticulate embodiment, the DAC inhibitor is romidepsin. In certain embodiments, the enhancer is sodium caprylate, sodium caprate, or sodium laurate. In certain particular embodiments, the enhancer is sodium caprate.

In the case of any of the above-mentioned embodiments, a controlled release coating may be applied to the final dosage form (capsule, tablet, multilayer tablet, multiparticulate composition, etc.). The controlled release coating may typically comprise a rate controlling polymer material as defined above. The dissolution characteristics of such a coating material may be pH dependent or independent of pH.

The various embodiments of the solid oral dosage forms of the invention may further comprise auxiliary excipient materials such as, for example, diluents, lubricants, disintegrants, plasticizers, anti-tack agents, wetting agents, surfactants, salts, opacifying agents, bulking agents, buffers, pigments, flavorings, and the like. As will be appreciated by those skilled in the art, the exact choice of excipients and their relative amounts will depend to some extent on the final dosage form.

Suitable diluents include, for example, pharmaceutically acceptable inert fillers such as sorbitol, microcrystalline cellulose, lactose, dibasic calcium phosphate, saccharides, and/or mixtures of any of the foregoing. Examples of diluents include, for example, sorbitol such as Parteck® SI 400 (Merck KGaA, Darmstadt, Germany), microcrystalline cellulose such as that sold under the Avicel trademark (FMC Corp., Philadelphia, Pa.), for example, Avicel™ pH101, Avicel™ pH102 and Avicel™ pH112; lactose such as lactose monohydrate, lactose anhydrous, and Pharmatose DCL21; dibasic calcium phosphate such as Emcompress® (JRS Pharma, Patterson, N.Y.); mannitol; starch; and sugars such as, for example, sucrose and glucose. Suitable lubricants, including agents that act on the flowability of the powder to be compressed are, for example, colloidal silicon dioxide such as Aerosil™ 200; talc; stearic acid, magnesium stearate, and calcium stearate. Suitable disintegrants include for example lightly cross-linked polyvinyl pyrrolidone, corn starch, potato starch, maize starch and modified starches, croscarmellose sodium, cross-povidone, sodium starch glycolate and combinations and mixtures thereof. Suitable wetting agents include polymers, carbohydrates, lipids, solvents, or small molecules including, but not limited to, alcohols and polyols such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, mono-, di- and trgycerides of medium chain fatty acids and derivatives thereof; glycerides cyclodextrins and cyclodextrin derivatives; ethers of polyethylene glycols having an average molecular weight of about 200 to about 6000, such as tetrahydrofurfuryl alcohol PEG ether or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide, and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof; and other solubilizers known in the art, such as dimethyl acetamide, dimethyl isosorbide, N-methylpyrrolidones, monooctanoin, diethylene glycol monoethyl ether, and water. In certain embodiments, the solubilizer is polyvinylpyrrolidone (PVP).

### EXAMPLE 1

### TRH Containing Tablets

(a) Caco-2 Monolayers.

Cell Culture: Caco-2 cells were cultured in Dulbecco's Modified Eagles Medium (DMEM) 4.5 g/L glucose supplemented with 1% (v/v) non-essential amino acids; 10% fetal calf serum and 1% penicillin/streptomycin. The cells were cultured at 37° C. and 5% CO₂ in 95% humidity. The cells were grown and expanded in standard tissue culture flasks and were passaged once they attained 100% confluence. The Caco-2 cells were then seeded on polycarbonate filter inserts (Costar; 12 mm diameter, 0.4 μm pore size) at a density of 5×10⁵ cells/cm² and incubated in six well culture plates with a medium change every second day. Confluent monolayers between day 20 and day 30 seeding on filters and at passages 30-40 were used throughout these studies.

Transepithelial Transport Studies: The effects of sodium salts of various MCFAs on the transport of ³H-TRH (apical to basolateral flux) was examined as follows: 15.0 μCi/ml (0.2 μM) ³3H-TRH was added apically at time zero for TRH flux experiments. The transport experiments were performed in Hank's Balanced Salt Solution (HBSS) containing 25 mM N-[2-hydroxyethyl]-piperazine-N′-[2-ethanesulfonic acid] (HEPES) buffer, pH 7.4 at 37° C. Due to variations in solubilities, various concentrations of the different MCFA sodium salts and various apical buffers were used as shown in Table 1. In all cases the basolateral chamber contained regular HBSS+HEPES.

TABLE 1
Concentrations and buffers used for various MCFA sodium salts
MCFA salt* Conc. (mM) Buffer

| | | |
|---|---|---|
| NaC8:0 | 0.32 | HBSS + HEPES |
| NaC10:0 | 0.40 | Ca²⁺ free HBSS |
| NaC12:0 | 3.77 | PBS** |
| NaC14:0 | 1.44 | PBS** |
| NaC18:0 | 0.16 | HBSS + HEPES |
| NaC18:2 | 0.16 | HBSS + HEPES |

*In the nomenclature CX:Y for a MCFA salt, X indicates the length of the carbon chain and Y indicates the position of unsaturation, if any.
**PBS—phosphate buffer solution.

After removing the cell culture medium, the monolayers were placed in wells containing pre-warmed HBSS (37° C.); 1 ml apically and 2 ml basolaterally. Monolayers were incubated at 37° C. for 30 minutes. Then at time zero, apical HBSS was replaced with the relevant apical test solution containing the radio-labeled compounds with and without the enhancer compound. Transepithelial electrical resistance (TEER) of the monolayer was measured at time zero and at 30 minute intervals up to 120 minutes using a Millicell ERS chopstix apparatus (Millipore (U.K.) Ltd., Hertfordshire, UK) with Evom to monitor the integrity of the monolayer. The plates were placed on an orbital shaker in an incubator (37° C.). Transport across the monolayers was followed by basolateral sampling (1 ml) at 30 minute intervals up to 120 minutes. At each 30-minute interval, each insert was transferred to a new well containing 2 ml fresh pre-warmed HBSS. Apical stock radioactivity was determined by taking 10 μl samples at t=0 and t=120 minutes. Scintillation fluid (10 ml) was added to each sample and the disintegrations per minute of each sample were determined in a Wallac System 1409 scintillation counter. Mean values for ³H-TRH concentrations were calculated for the apical and basolateral solutions at each time point. The apparent permeability coefficients were calculated using the method described by Artursson (see Artursson P., J. Pharm. Sci. 79:476-482 (1990)).

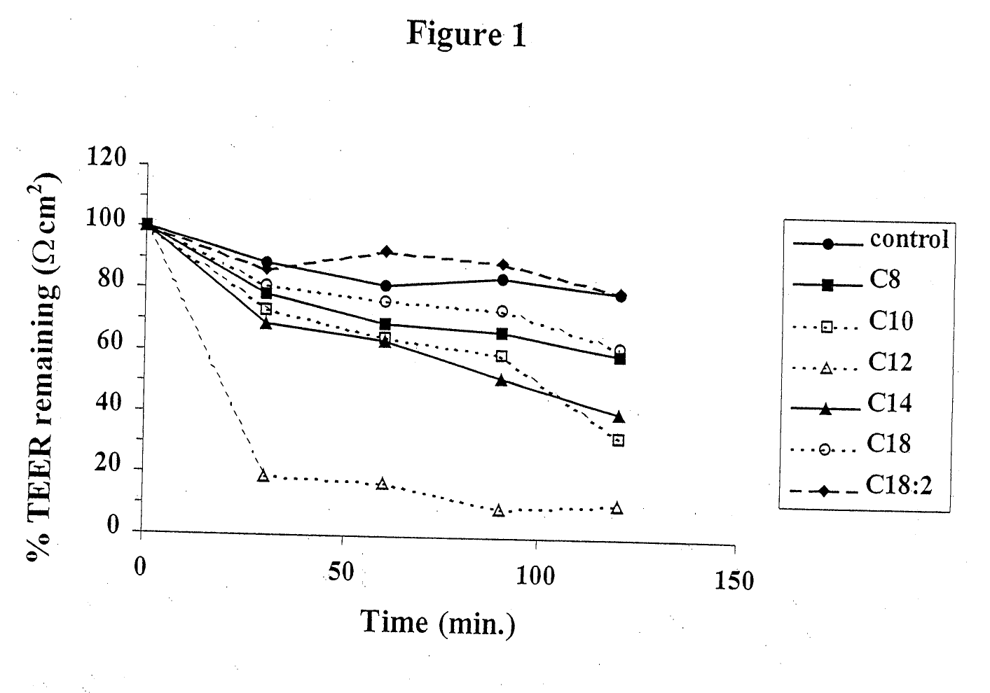

FIG. 1 shows the effect of C8, C10, C12, C14, C18, and C18:2 sodium salts with 3H-TRH on TEER (Ωcm²) in Caco-2 monolayers over 2 hours. The data for the C8, C10, C14, and C18 indicate minimal reduction in TEER compared to the control. While the data for C12 indicates some cell damage (reduction in TEER), this reduction is probably a result of the higher concentration of enhancer used in this.

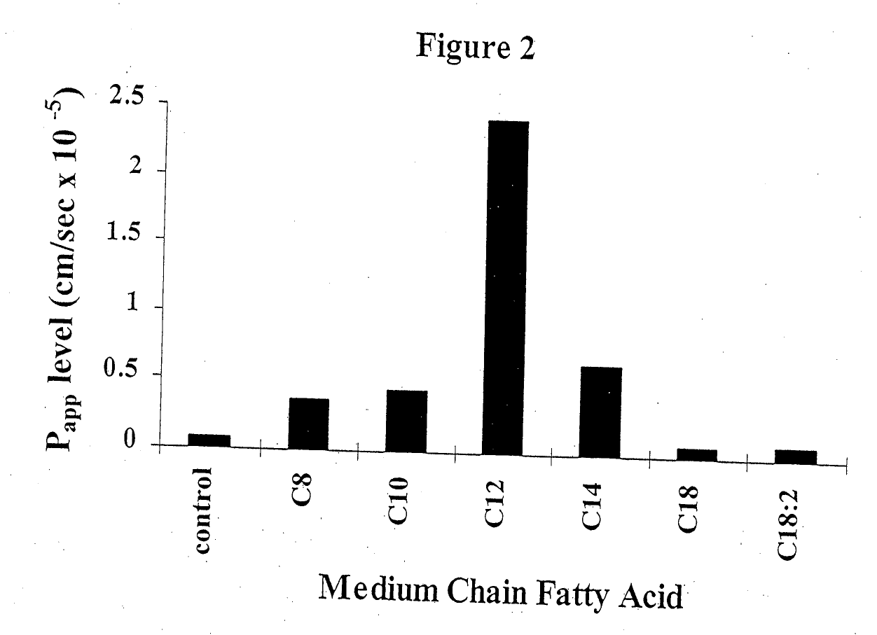

FIG. 2 shows the effect of C8, C10, C12, C14, C18, and C18:2 sodium salts on Pₐₚₚ for ³H-TRH across in Caco-2 monolayers. Compared to the control, the sodium salts of C8, C10, C12, and C14 showed considerable increases in the permeability constant, Pₐₚₚ, at the concentrations used. It is noted that the high Pₐₚₚ value observed for the C12 salt may be indicative of cell damage at this high enhancer concentration.

Mitochondrial Toxicity Assay: Mitochondrial dehydrogenase (MDH) activity was assessed as a marker of cell viability using a method based on the color change of tetrazolium salt in the presence MDH. Cells were harvested, counted, and seeded on 96 well plates at an approximate density of 10⁶ cells/ml (100 μl of cell suspension per well). The cells were then incubated at 37° C. for 24 hours in a humidified atmosphere with 5% CO₂. A number of wells were treated with each MCFA sodium salt solution at the concentrations shown in Table 1, and the plate was incubated for 2 hours. After incubation 10 μl of MTT labeling reagent was added to each well for 4 hours. Solubilization buffer (100 μl; see Table 1) was added to each well, and the plate was incubated for a further 24 hours. Absorbance at 570 nm of each sample was measured using a spectrophotometer (Dynatech MR7000).

(b) In Vivo Administration (Closed Loop Rat Model).

In vivo rat closed loop studies were modified from the methods of Doluisio et al. (see Doluisio J. T., et al.: Journal of Pharmaceutical Science (1969), 58, 1196-1200) and Brayden et al. (see Brayden D.: Drug Delivery Pharmaceutical News (1997) 4(1)). Male Wistar rats (weight range 250 g-350 g) were anaesthetized with ketamine hydrochloride/acepromazine. A mid-line incision was made in the abdomen and a segment of the duodenum (7-9 cm of tissue) was isolated about 5 cm distal from the pyloric sphincter, taking care to avoid damage to surrounding blood vessels. The sample solutions (PBS containing C8 or C10 (35 mg) and TRH (500 μg and 1000 μg)) and control (PBS containing TRH only (500 μg and 1000 μg)) warmed to 37° C. were administered directly into the lumen of the duodenal segment using a 26 G needle. All intraduodenal dose volumes (for samples and control) were 1 ml/kg. The proximal end of the segment was ligated, and the loop was sprayed with isotonic saline (37° C.) to provide moisture and then replaced in the abdominal cavity avoiding distension. The incision was closed with surgical clips. A group of animals were administered TRH in PBS (100 μg in 0.2 ml) by subcutaneous injection as a reference.

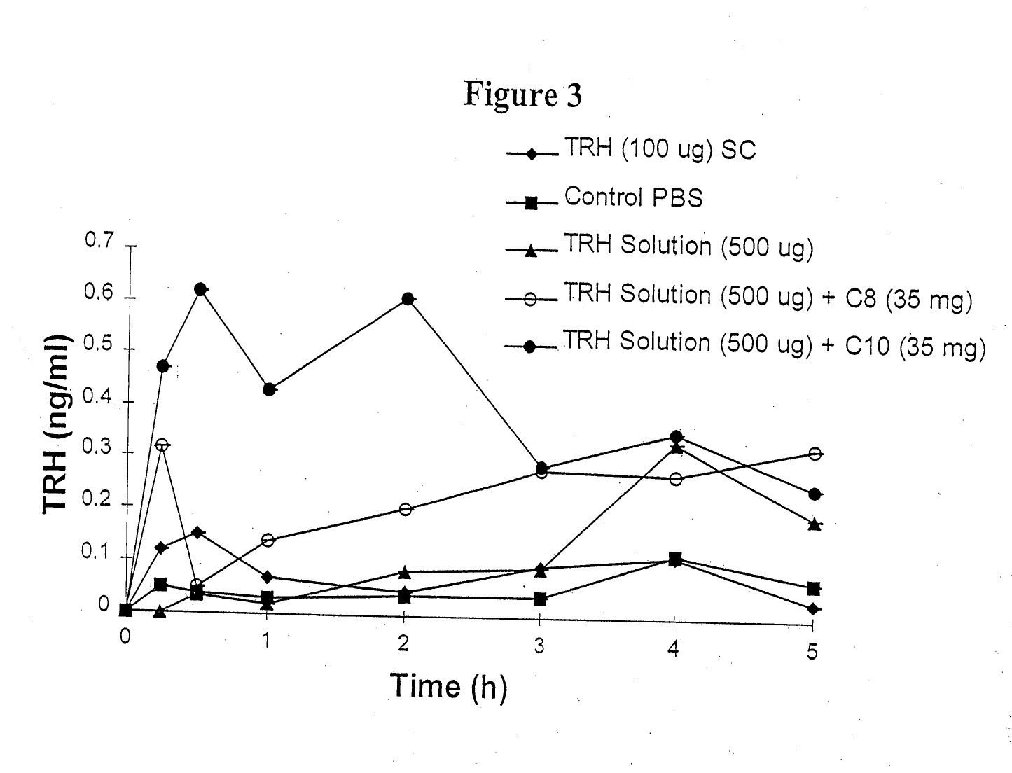

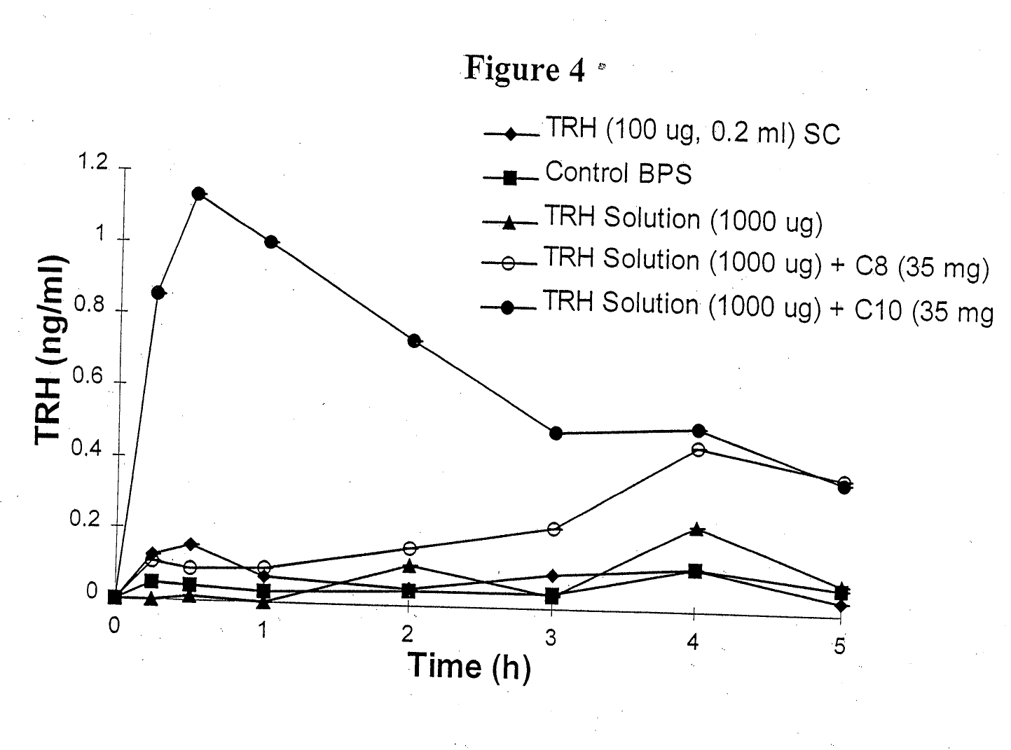

FIG. 3 shows the serum TRH concentration-time profiles following interduodenal bolus dose of 500 μg TRH with NaC8 or NaC10 (35 mg) enhancer present, according to the closed loop rat model. FIG. 4 shows the serum TRH concentration-time profiles following interduodenal bolus dose of 1000 μg TRH with NaC8 or NaC10 (35 mg) enhancer present, according to the closed loop rat model. From FIGS. 3 and 4 it can be seen that the presence of the enhancer in each case significantly increases the serum levels of TRH over the control TRH solution indicating increased absorption of the drug in the presence of the enhancer.

(c) Tableting.

Having established the enhancing effect of NaC8 and NaC10 on TRH in solution, immediate release (IR) and sustained release (SR) TRH tablets and the like may be prepared. IR and SR formulations are detailed in Tables 2 and 3 below.

TABLE 2
THR IR tablet formulation details (all amounts in wt %)

| | | | Silica | Mag. | | Disinte- | Micro. | |
|---|---|---|---|---|---|---|---|---|
| TRH | NaC₈ | NaC10 | Dioxide | Stearate | Lactose | Grant | Cellulose | PVP |
| 0.64 | 70.36 | — | 0.5 | 0.5 | 20 | 8 | — | — |
| 1.27 | 69.73 | — | 0.5 | 0.5 | 20 | 8 | — | — |
| 1.23 | — | 67.64 | 0.5 | 0.5 | 20 | 8 | — | 2.13 |
| 2.42 | — | 66.45 | 0.5 | 0.5 | — | 8 | 20 | 2.13 |
| 2.42 | — | 66.45 | 0.5 | 0.5 | 20 | 8 | — | 2.13 |

TABLE 3
THR SR tablet formulation details (all amounts in wt. %)

| | | | | | Micro- | |
|---|---|---|---|---|---|---|
| | | Silica | Magnesium | | crystalline | |
| TRH | NaC₁₀ | Dioxide | Stearate | HPMC^{(a)} | Cellulose | PVP |
| 1.41 | 77.59 | 0.5 | 0.5 | 20 | — | — |
| 1.05 | 57.95 | 0.5 | 0.5 | 20 | 20 | — |
| 2.68 | 73.94 | 0.5 | 0.5 | 20 | — | 2.37 |

### EXAMPLE 2

### Heparin Containing Tablets

(a) Closed-loop Rat Segment.

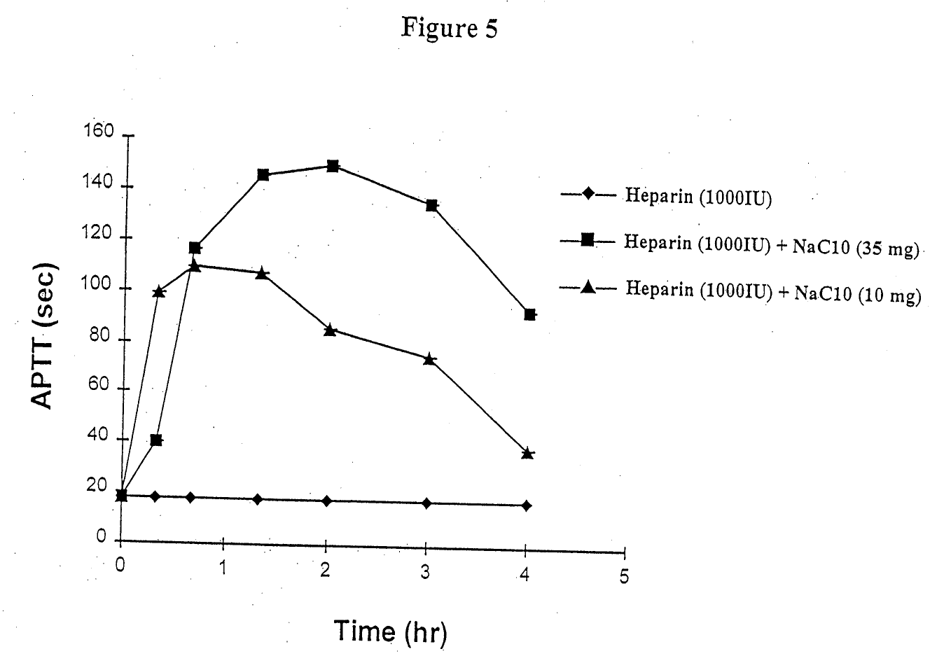

The procedure carried out in Example 1 (a) above was repeated using USP heparin in place of TRH and dosing intraileally rather than intraduodenally. A mid-line incision was made in the abdomen and the distal end of the ileum located (about 10 cm proximal to the ileo-caecal junction). 7-9 cm of tissue was isolated and the distal end ligated, taking care to avoid damage to surrounding blood vessels. Heparin absorption as indicated by activated prothrombin time (APTT) response was measured by placing a drop of whole blood (freshly sampled from the tail artery) on the test cartridge of a Biotrack 512 coagulation monitor. APTT measurements were taken at various time points. FIG. 5 shows the APTT response of USP heparin (1000 iu) at different sodium caprate (C10) levels (10 and 35 mg). Using APTT response as an indicator of heparin absorption into the bloodstream, it is clear that there is a significant increase in absorption in the presence of sodium caprate compared to the control heparin solution containing no enhancer.

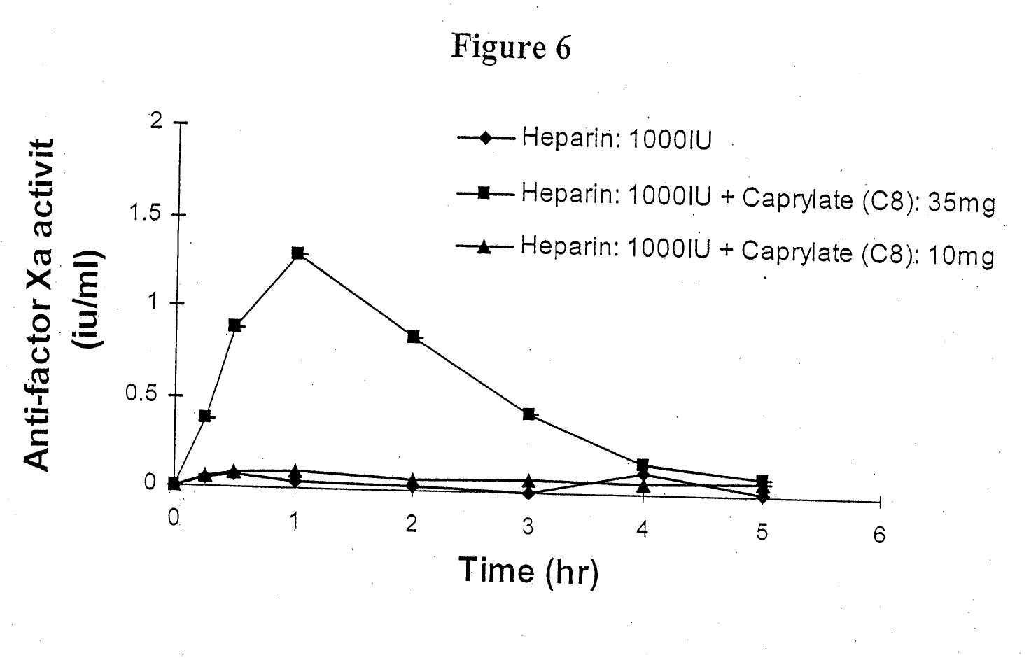

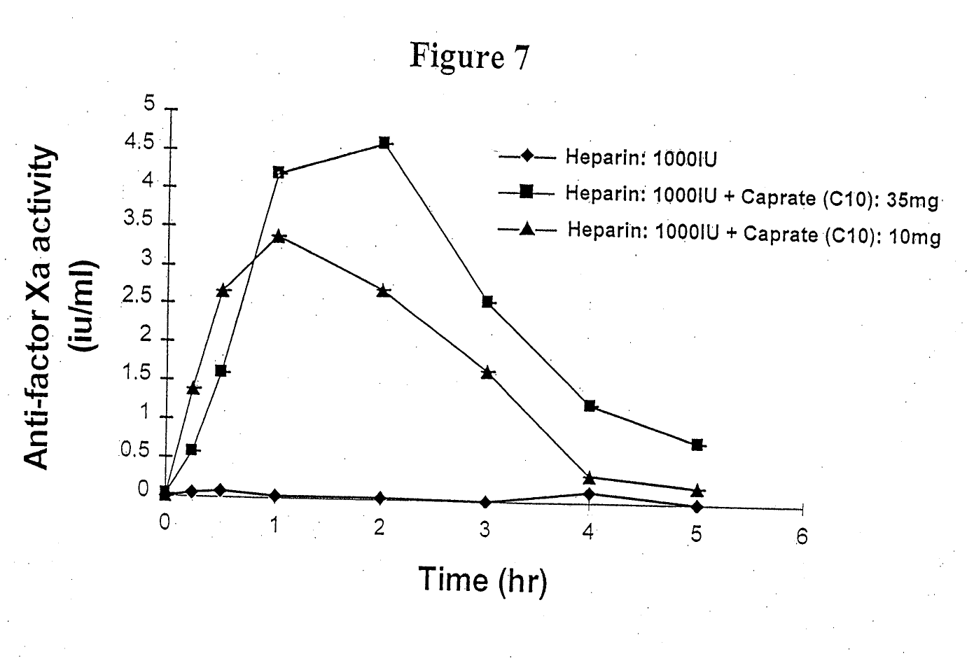

Citrated blood samples were centrifuged at 3000 rpm for 15 mins. to obtain plasma for anti-factor Xₐ analysis. FIG. 6 shows the anti-factor Xₐ response of USP heparin (1000 iu) in the presence of sodium caprylate (C8, 10 mg and 35 mg). FIG. 7 shows the anti-factor Xₐ response of USP heparin (1000 iu) in the presence of sodium caprate (C10, 10 mg and 35 mg). The control in each case is a solution of the same heparin concentration containing no enhancer. The significant increase in anti-factor Xₐ activity observed for NaC8 (at 35 mg dose) and NaC10 (at both 10 mg and 35 mg doses) is indicative of the increase in heparin absorption relative to the control heparin solution.

(b) Tableting.

(i) IR Tablets.

Instant release (IR) tablets containing heparin sodium USP (197.25 IU/mg, supplied by Scientific Protein Labs., Waunkee, Wis.) and an enhancer (sodium caprylate, NaC8; sodium caprate, NaC10, supplied by Napp Technologies, New Jersey) were prepared according to the formulae detailed in Table 4 by direct compression of the blend using a Manesty (E) single tablet press. The blend was prepared as follows: heparin, the enhancer, and tablet excipients (excluding where applicable colloidal silica dioxide and magnesium stearate) were weighed out into a container. The colloidal silica dioxide, when present, was sieved through a 425 μm sieve into the container, after which the mixture was blended for four minutes before adding the magnesium stearate and blending for a further one minute.

TABLE 4
Formulation data for IR tablets containing
heparin and enhancer (all amounts in wt. %)

| Batch | | | | Silica | Magnesium | | Disinte- | |
|---|---|---|---|---|---|---|---|---|
| No. | NaC₈ | NaC₁₀ | Heparin | dioxide | stearate | Mannitol | grant^{(a)} | PVP^{(b)} |
| 1 | 65.7 | — | 13.3 | 0.5 | 0.5 | 20.0 | — | — |
| 2 | 62.2 | — | 16.8 | 0.5 | 0.5 | 20.0 | — | — |
| 3 | 57.49 | — | 21.91 | 0.1 | 0.5 | 20.0 | — | — |
| 4 | 75.66 | — | 15.34 | 0.5 | 0.5 | — | 8.0 | — |
| 5 | — | 62.0 | 37.5 | 0.5 | — | — | — | — |
| 6 | — | 49.43 | 30.07 | 0.5 | — | 20.0 | — | — |
| 7 | — | 31.29 | 25.94 | 0.5 | 0.5 | 40.0 | — | 1.77 |

“—“ indicates “not applicable”
^{(a)}Disintegrant used was sodium starch glycolate;
^{(b)}PVP = polyvinyl pyrrolidone

The potency of tablets prepared above was tested using a heparin assay based on the azure dye determination of heparin. The sample to be assayed was added to an Azure A dye solution and the heparin content was calculated from the absorbance of the sample solution at 626 nm. Tablet data and potency values for selected batches detailed in Table 4 are given in Table 5. Dissolution profiles for IR tablets according to this Example in phosphate buffer at pH 7.4 were determined by heparin assay, sampling at various time points.

Heparin/sodium caprylate: Tablets from batches 1 and 2 gave rapid release yielding 100% of the drug at 15 minutes. Tablets from batch 4 also gave rapid release yielding 100% release at 30 minutes.

Heparin/sodium caprate: Tablets from batches 5 and 6 gave rapid release of 100% of the drug at 15 minutes.

TABLE 5
Tablet data and potency values for IR heparin tablets

| | | Tablet | | | Actual heparin | Potency |
|---|---|---|---|---|---|---|
| Batch | | Weight | Hardness | Disintegration | Potency | As % of |
| No. | Enhancer | (mg) | (N) | Time(s) | (mg/g) | Label |
| 1 | NaC8 | 431 ± 5 | 85 ± 4 | — | 145.675 | 109 |
| 2 | NaC₈ | 414 ± 14 | 82 ± 9 | — | 175.79 | 105 |
| 3 | NaC₈ | 650 ± 4 | 71 ± 12 | 552 | 166.4 | 119 |
| 4 | NaC₈ | 377 ± 2 | 58 ± 10 | — | 168.04 | 110 |
| 5 | NaC₁₀ | 408 ± 21 | 79 ± 7 | — | 394.47 | 105 |
| 6 | NaC₁₀ | 490 ± 6 | 124 ± 10 | — | 323.33 | 108 |
| 7 | NaC₁₀ | 584 ± 12 | 69 ± 22 | 485 | 143.0 | 102 |

(ii) SR Tablets.

Using the same procedure as used in (i) above, sustained release (SR) tablets were prepared according to the formulae shown in Table 6. The potency of controlled release tablets was determined using the same procedure as in (i) above. Tablet details and potency for selected batches are shown in Table 7. Dissolution profiles for SR tablets according this Example were determined by heparin assay at pH 7.4, sampling at various time points.

Heparin/sodium caprylate: Dissolution data for batches 8, 9, and 11 are shown in Table 8. From this data it can be seen that heparin/sodium caprylate SR tablets with 15% Methocel K100LV with and without 5% sodium starch glycolate (batches 8 & 9) gave a sustained release with 100% release occurring between 3 and 4 hours. Batch 11 sustaining 10% mannitol gave a faster release.

Heparin/sodium caprate: Dissolution data for batches 13 and 14 are shown in Table 8. From these data it can be seen that heparin/sodium caprate SR tablets with 20% Methocel K100LV (batch 13) demonstrated a sustained release of the drug compound over a six-hour period. Where Methocel K15M (batch 14) was used in place of Methocel K100LV, release of the drug compound was incomplete after 8 hours.

TABLE 6
Formulation data for SR tablets containing heparin and enhancer (all amounts in wt. %)

| Batch | | | | Silica | Mg. | | | | Micro. | |
|---|---|---|---|---|---|---|---|---|---|---|
| No. | NaC₈ | NaC¹⁰ | Heparin | dioxide | stearate | HPMC^{(a)} | Disintegrant^{(b)} | Mannitol | cellulose | PVP^{(c)} |
| 8 | 69.84 | — | 14.16 | 0.5 | 0.5 | 15 | — | — | — | — |
| 9 | 65.68 | — | 13.32 | 0.5 | 0.5 | 15 | 5.0 | — | — | — |
| 10 | 65.68 | — | 13.32 | 0.5 | 0.5 | 12 | 8.0 | — | — | — |
| 11 | 65.68 | — | 13.32 | 0.5 | 0.5 | 10.0 | — | 10.0 | — | — |
| 12 | 53.77 | — | 20.48 | — | 1.0 | 14.85 | — | — | 9.9 | — |
| 13 | — | 56.2 | 23.3 | 0.5 | — | 20.0 | — | — | — | — |
| 14 | — | 56.2 | 23.3 | 0.5 | — | 20.0* | — | — | — | — |
| 15 | — | 41.63 | 34.52 | 0.5 | 1.0 | 20.0 | — | — | — | 2.35 |

“—” indicates ″not applicable″;
^{(a)}Hydroxypropylmethyl cellulose: Methocel K100LV in each case except ″*″ in which Methocel K15M was employed;
^{(b)}Disintegrant used was sodium starch glycolate;
^{(c)}PVP = polyvinyl pyrrolidone;

TABLE 7
Table data and Potency values for SR heparin tablets

| | | Tablet | Hardness | Disintegration | Actual Heparin |
|---|---|---|---|---|---|
| Batch No. | Enhancer | Weight (mg) | (N) | Time (s) | potency (mg/g) |
| 8 | NaC₈ | 397 ± 5 | 52 ± 11 | — | — |
| 9 | NaC₈ | 436 ± 11 | 40 ± 10 | — | 140.08 |
| 10 | NaC₈ | 384 ± 4 | 42 ± 12 | — | — |
| 11 | NaC₈ | 400 ± 8 | 72 ± 16 | — | 129.79 |
| 12 | NaC₈ | 683 ± 9 | 84 ± 17 | 3318 | 147.10 |
| 13 | NaC₁₀ | 491 ± 14 | 69 ± 7 | — | — |
| 14 | NaC₁₀ | 456 ± 13 | 47 ± 4 | — | — |
| 15 | NaC₁₀ | 470 ± 29 | — | 2982 | 148.20 |

TABLE 8
Dissolution data for selected batches of SR tablets

| | % Release (as of label) | | | | |
|---|---|---|---|---|---|
| Time | Batch 8 | Batch 9 | Batch 11 | Batch 13 | Batch 14 |
| (min) | (NaC₈) | (NaC₈) | (NaC₈) | (NaC₁₀) | (NaC₁₀) |
| 0 | 0 | 0 | 0 | 0 | 0 |
| 15 | 22.9 | 21.2 | 45.3 | 18.8 | 5.7 |
| 30 | 37.3 | 30.8 | 72.3 | 45.0 | 11.6 |
| 60 | 57.8 | 54.5 | 101.9 | 44.8 | 11.2 |
| 120 | 92.2 | 90.8 | 109.4 | 65.2 | 20.0 |
| 240 | 109.5 | 105.8 | 96.4 | 83.1 | 33.9 |
| 360 | — | — | — | 90.3 | 66.0 |
| 480 | — | — | — | 102.7 | 82.8 |

(iii) Enteric Coated Tablets.

Tablets from batches 7 and 15 were enterically coated with a coating solution as detailed in Table 9. Tablets were coated with 5% w/w coating solution using a side vented coating pan (Freund Hi-Coater). Disintegration testing was carried out in a VanKel disintegration tester VK100E4635. Disintegration medium was initially simulated gastric fluid pH 1.2 for one hour and then phosphate buffer pH 7. The disintegration time recorded was the time from introduction into phosphate buffer pH 7.4 to complete disintegration. The disintegration time for enterically coated tablets from batch 7 was 34 min. 24 sec., while for enteric coated tablets from batch 15 the disintegration time was 93 min. 40 sec.

TABLE 9
Enteric coating solution

| Component | Amount (wt. %) |
|---|---|
| Eudragit ® 12.5 | 49.86 |
| Diethylphthlate | 1.26 |
| Isopropyl alcohol | 43.33 |
| Talc | 2.46 |
| Water | 3.06 |

(c) Dog Study.

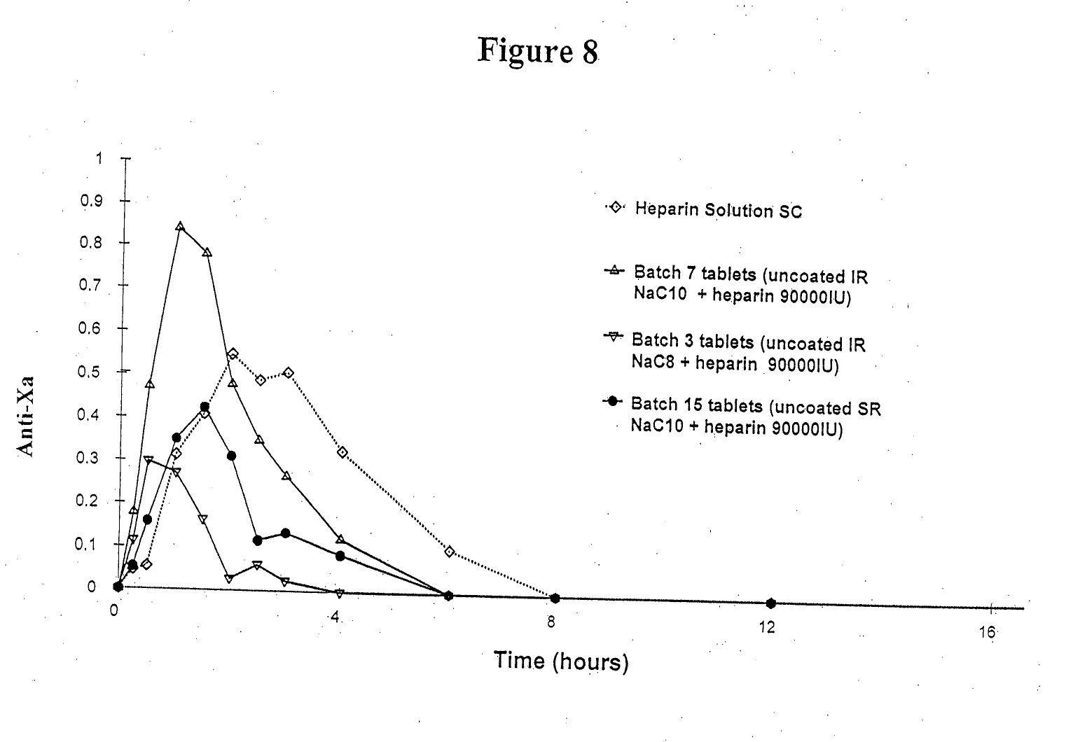

Tablets from batches 3, 7 and 15 in Tables 5 and 6 above were dosed orally to groups of five dogs in a single dose crossover study. Each group was dosed with (1) orally administered uncoated IR tablets containing 90000 IU heparin and 550 mg NaC10 enhancer (batch 7); (2) orally administered uncoated IR tablets containing 90000 IU heparin and 550 mg NaC8 enhancer (batch 3); (3) orally administered uncoated SR tablets containing 90000 IU heparin and 550 mg NaC10 enhancer (batch 15); and (4) s.c. administered heparin solution (5000 IU, control). Blood samples for anti-factor Xₐ analysis were collected from the jugular vein at various time points. Clinical assessment of all animals pre- and post-treatment indicated no adverse effects on the test subjects. FIG. 8 shows the mean anti-factor Xₐ response for each treatment, together with the s.c. heparin solution reference. The data in FIG. 8 shows an increase in the plasma anti-factor Xₐ activity for all of the formulations according to the invention. This result indicates the successful delivery of bioactive heparin using both NaC8 and NaC10 enhancers. Using IR formulations and an equivalent dose of heparin, a larger anti-factor Xₐ response was observed with the NaC10 enhancer, in spite of the lower dose of NaC10 relative to NaC8 administered (NaC10 dose was half that of NaC8). The anti-factor Xₐ response can be sustained over longer time profiles relative to IR formulations by the use of SR tablets.

### EXAMPLE 3

### Effect of Enhancers on the Systemic Availability of Low Molecular Weight Heparin (LMWH) after Intraduodenal Administration in Rats

Male Wistar rats (250 g-350 g) were anaesthetized with a mixture of ketamine hydrochloride (80 mg/kg) and acepromazine maleate (3 mg/kg) given by intra-muscular injection. The animals were also administered with halothane gas as required. A midline incision was made in the abdomen and the duodenum was isolated. The test solutions, comprising parnaparin sodium (LMWH) (Opocrin SBA, Modena, Italy) with or without enhancer reconstituted in phosphate buffered saline (pH 7.4), were administered (1 ml/kg) via a cannula inserted into the intestine approximately 10-12 cm from the pyloris. The intestine was kept moist with saline during this procedure. Following drug administration, the intestinal segment was carefully replaced into the abdomen, and the incision was closed using surgical clips. The parenteral reference solution (0.2 ml) was administered subcutaneously into a fold in the back of the neck.

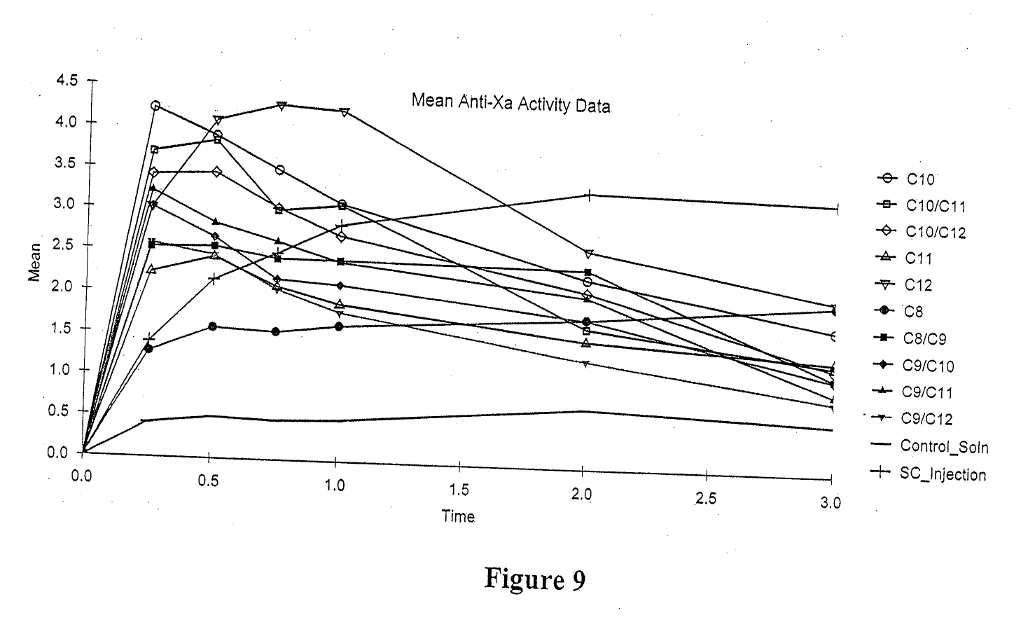

Blood samples were taken from a tail artery at various intervals and plasma anti-factor Xₐ activity was determined. FIG. 9 shows the mean anti-factor Xₐ response over a period of 3 hours following intraduodenal administration to rats of phosphate buffered saline solutions of parnaparin sodium (LMWH) (1000 IU), in the presence of 35 mg of different enhancers [sodium caprylate (C8), sodium nonanoate (C9), sodium caprate (C10), sodium undecanoate (C11), sodium laurate (C12)] and different 50:50 binary mixtures of enhancers, to rats (n=8) in an open loop model. The reference product comprised administering 250 IU parnaparin sodium subcutaneously. The control solution comprised administering a solution containing 1000 IU parnaparin sodium without any enhancer intraduodenally.

FIG. 9 shows that the systemic delivery of LMWH in the absence of enhancer is relatively poor after intraduodenal administration to rats; however, the co-administration of the sodium salts of medium chain fatty acids significantly enhanced the systemic delivery of LMWH from the rat intestine

### EXAMPLE 4

### Effect of Enhancers on the Systemic Availability of Leuprolide after Intraduodenal Administration in Dogs

Beagle dogs (10-15 Kg) were sedated with medetomidine (80 μg/kg) and an endoscope was inserted via the mouth, esophagus, and stomach into the duodenum. The test solutions (10 ml) comprising leuprolide acetate (Mallinckrodt Inc, St. Louis, Mo.) with or without enhancer reconstituted in deionized water were administered intraduodenally via the endoscope. Following removal of the endoscope, sedation was reversed using atipamezole (400 μg/kg). The parenteral reference solutions comprising 1 mg Leuprolide reconstituted in 0.5 ml sterile water were administered intravenously and subcutaneously respectively.

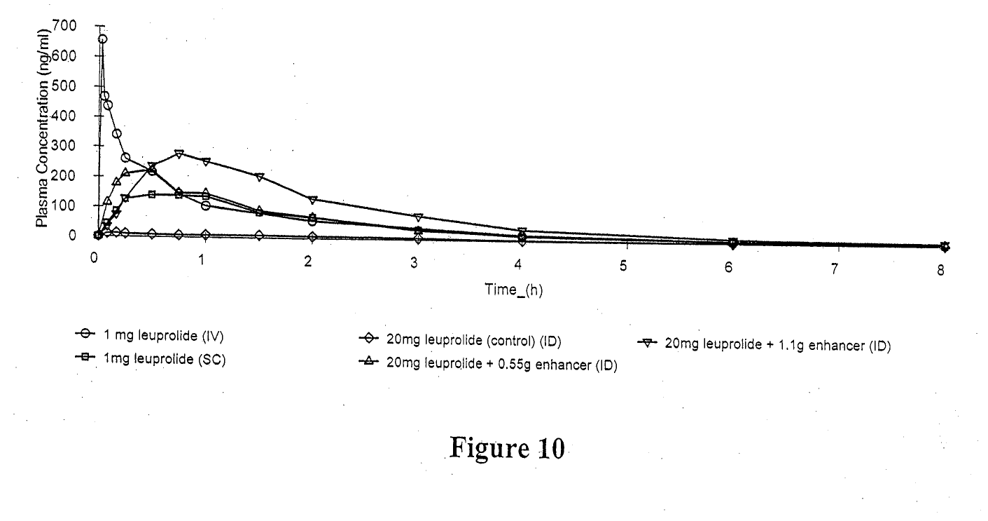

Blood samples were taken from the jugular vein at various intervals and plasma leuprolide levels were determined. The resulting mean plasma leuprolide levels are shown in FIG. 10. The results show that, although the systemic delivery of leuprolide when administered intraduodenally without enhancer is negligible, coadministration with enhancer resulted in a considerable enhancer dose dependent enhancement in the systemic delivery of leuprolide; a mean % relative bioavailability of 8% observed for at the upper dose of enhancer.

### EXAMPLE 5

### Effect of Enhancers on the Systemic Availability of LMWH after Oral Administration in Dogs

(a) Granulate Manufacture

A 200 g blend containing parnaparin sodium (47.1%), sodium caprate (26.2%), mannitol (16.7%), and Explotab™ (Roquette Freres, Lestrem, France) (10.0%) was granulated in a Kenwood Chef mixer using water as the granulating solvent. The resulting granulates were tray dried in an oven at 67-68° C. and size reduced through 1.25 mm, 0.8 mm, and 0.5 mm screens respectively in an oscillating granulator. The actual potency of the resulting granulate was determined as 101.1% of the label claim.

(b) 30,000 IU LMWH/183 mg Sodium Caprate Instant Release Tablet Manufacture

The granulate described above was bag blended with 0.5% magnesium stearate for 5 minutes. The resulting blend was tableted using 13 mm round concave tooling on a Riva Piccalo tablet press to a target tablet content of 30,000 IU parnaparin sodium and 183 mg sodium caprate. The tablets had a mean tablet hardness of 108 N and a mean tablet weight of 675 mg. The actual LMWH content of the tablets was determined as 95.6% of label claim.

Disintegration testing was carried out on the tablets. One tablet was placed in each of the six tubes of the disintegration basket. The disintegration apparatus was operated at 29-30 cycles per minute using de-ionized water at 37° C. Tablet disintegration was complete in 550 seconds.

(c) 90,000 IU LMWH/0.55 g Sodium Caprate Solution Manufacture

90,000 IU parnaparin sodium and 0.55 g sodium caprate were individually weighed into glass bottles and the resulting powder mixture was reconstituted with 10 ml water.

(d) Dog Biostudy Evaluation

90,000 IU parnaparin sodium and 550 mg sodium caprate was administered as both a solution dosage form (equivalent to 10 ml of the above solution composition) and a fast disintegrating tablet dosage form (equivalent to 3 tablets of the above tablet composition) in a single dose, non randomized, cross-over study in a group of six female beagle dogs (9.5-14.4 Kg) with a seven day washout between treatments. A subcutaneous injection containing 5000 IU parnaparin sodium was used as the reference.

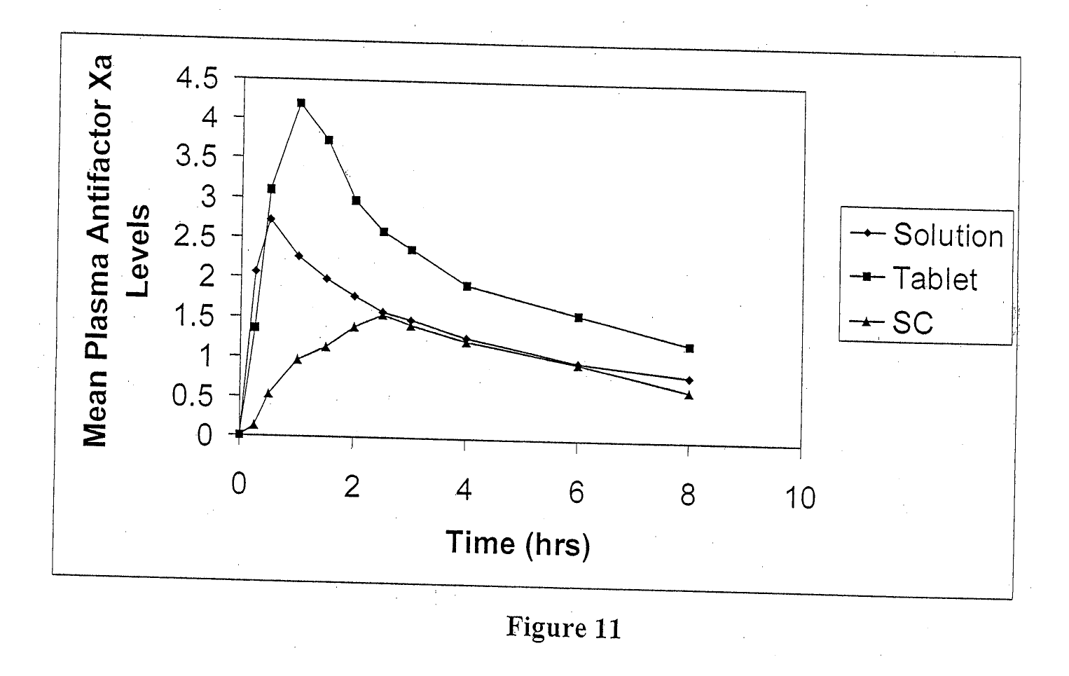

Blood samples were taken from the jugular vein at various intervals and anti-factor Xₐ activity was determined. Data was adjusted for baseline anti-factor Xₐ activity. The resulting mean plasma anti-factor Xₐ levels are summarized in FIG. 11. Both the tablet and solution dosage forms showed good responses when compared with the subcutaneous reference leg. The mean delivery, as determined by plasma antifactor Xₐ levels, of parnaparin sodium from the solid dosage form was considerably greater than that from the corresponding solution dosage form.

### EXAMPLE 6

### Effect of Enhancers on the Systemic Availability of LMWH after Oral Administration in Humans

(a) Granulate Manufacture

Parnaparin sodium (61.05%), sodium caprate (33.95%), and polyvinyl pyrrolidone (Kollidon 30, BASF AG, Ludwigshafen, Germany) (5.0%) were mixed for 5 minutes in a Gral 10 prior to the addition of water, which was then gradually added, with mixing, using a peristaltic pump until all the material was apparently granulated.

The resultant granulates were tray dried in an oven at either 50° C. for 24 hours. The dried granules were milled through a 30 mesh screen using a Fitzmill M5A

(b) 45,000 IU LMWH/275 mg Sodium Caprate Instant Release Tablet Manufacture

The parnaparin sodium/sodium caprate/polyvinyl pyrrolidone granulate (78.3%) was blended for 5 minutes with mannitol (16.6%), Explotab (5.0%), and magnesium stearate (1.0%) in a 10 liter V Cone blender. The potency of the resulting blend (480.41 mg/g) was 100.5% of the label claim. The blend was tableted using 13 mm round normal concave tooling on the Piccola 10 station press in automatic mode to a target content of 45,000 IU LMWH and 275 mg sodium caprate. The resulting instant release tablets had a mean tablet weight of 1027 mg, a mean tablet hardness of 108 N and a potency of 97% label claim. The tablets showed a disintegration time of up to 850 seconds and 100% dissolution into pH 1.2 buffer in 30 minutes.

(c) 90,000 IU LMWH/550 mg Sodium Caprate Solution Manufacture

Two instant tablets, each containing 45,000 IU LMWH and 275 mg sodium caprate, were reconstituted in 30 ml water.

(d) Human Biostudy Evaluation

90,000 IU LMWH and 550 mg sodium caprate was orally administered to 12 healthy human volunteers as both a solution dosage form (equivalent to 30 ml of the above solution dosage form) and as a solid dosage form (equivalent to 2 tablets of the above composition) in an open label, three treatment, three period study with a seven day washout between each dose; Treatments A (Instant Release Tablets) and B (Oral Solution) were crossed over in a randomized manner whereas Treatment C (6,400 IU Fluxum™ SC (Hoechst Marion Roussel), a commercially available injectable LMWH product) was administered to the same subjects as a single block.

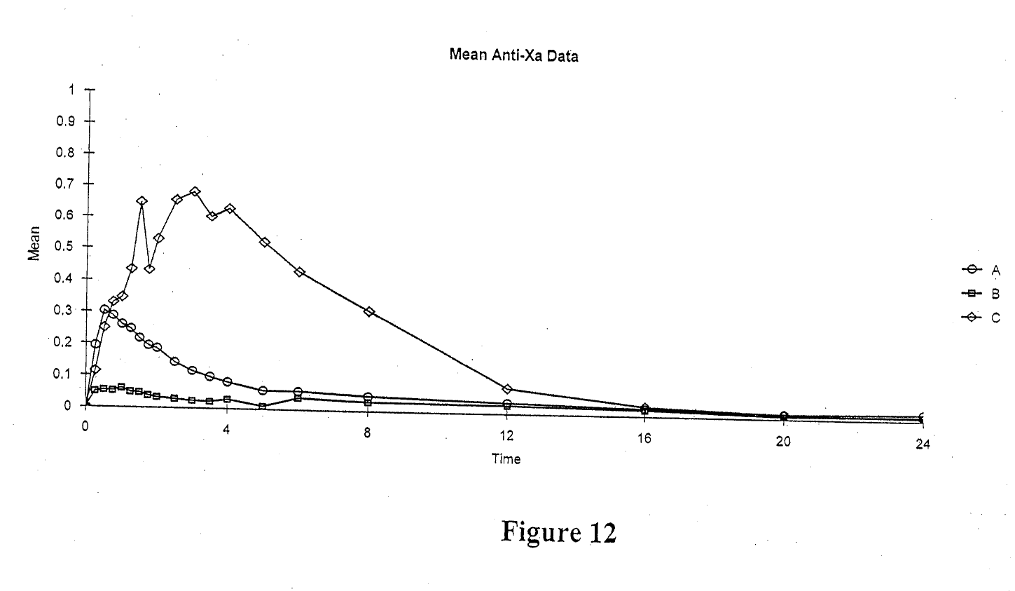

Blood samples were taken at various intervals and anti-factor Xₐ activity was determined. The resulting mean anti-factor Xₐ levels are shown in FIG. 12. Treatments A and B exhibited unexpectedly low responses when compared with the subcutaneous reference treatment. It should be noted, however, that the mean delivery of LMWH, as measured by plasma anti-factor Xₐ levels, was considerably higher from the solid dosage form than that from the corresponding solution dosage form for which a mean % bioavailability of only 0.9% was observed.

### EXAMPLE 7

Effect of Enhancers on the Systemic Availability of LMWH after Intrajejunal Administration in Humans

(a) Solution Manufacture

The following LMWH/sodium caprate combinations were made with 15 ml deionized water:

(i) 20,000 IU LMWH, 0.55 g Sodium Caprate;

(ii) 20,000 IU LMWH, 1.1 g Sodium Caprate;

(iii) 45,000 IU LMWH, 0.55 g Sodium Caprate;

(iv) 45,000 IU LMWH, 1.1 g Sodium Caprate;

(v) 45,000 IU LMWH, 1.65 g Sodium Caprate.

(b) Human Biostudy Evaluation

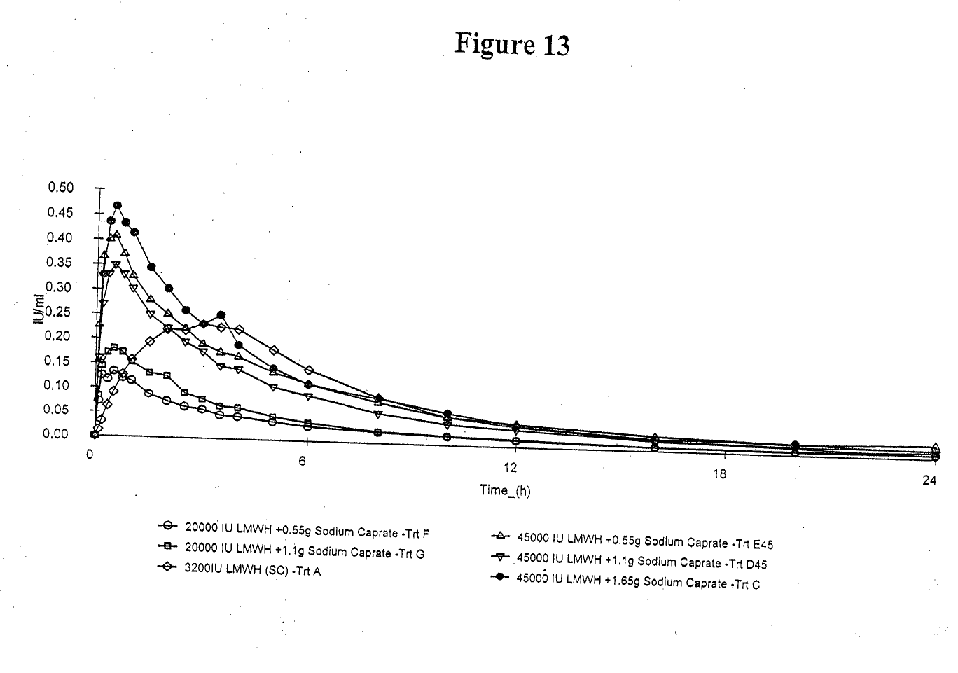

15 ml of each of the above solutions was administered intrajejunally via a nasojejunal intubation in an open label, six treatment period crossover study in up to 11 healthy human volunteers. 3,200 IU Fluxum™ SC was included in the study as a subcutaneous reference. Blood samples were taken at various intervals and anti-factor Xₐ activity was determined. The resulting mean anti-factor Xₐ levels are shown in FIG. 13.

It should be noted that the mean % relative bioavailability for each treatment in the current study was considerably higher than the mean % bioavailability observed for the solution dosage form in Example 6; mean % bioavailabilities ranging from 5% to 9% were observed for the treatments in the current study suggesting that the preferred LMWH oral dosage form containing sodium caprate should be designed to minimize release of drug and enhancer in the stomach and maximize the release of drug and enhancer in the small intestine.

### EXAMPLE 8

### Manufacture of Delayed Release Tablet Dosage Form Containing LMWH and Enhancer

(a) LMWH/Sodium Caprate Granulate Manufacture

A 500 g batch of parnaparin sodium:sodium caprate (0.92:1) was granulated in a Gral 10 using a 50% aqueous solution of Kollidon 30 as the granulating solvent. The resulting granulate was dried for 60 minutes in a Niro Aeromatic Fluidized Bed Drier at a final product temperature of 25° C. The dried granulate was milled through a 30 mesh screen in a Fitzmill M5A. The potency of the resulting dried granulate was determined as 114.8% of the label claim.

(b) 22,500 IU LMWH/275 mg Sodium Caprate Instant Release Tablet Manufacture

The above granulate (77.5%) was added to mannitol (16%), Polyplasdone™ XL (ISP, Wayne, N.J.) (5%) and Aerosil™ (1%) (Degussa, Rheinfelden, Germany) in a 10 IV coned blender and blended for 10 minutes. Magnesium stearate (0.5%) was added to the resulting blend and blending was continued for a further 3 minutes. The resulting blend was tableted on Piccola tablet press using 13 mm round normal concave tooling to a mean tablet weight of 772 mg and a mean tablet hardness of 140 N. The actual potency of the resulting tablets was determined as 24,017 IU LMWH per tablet.

(c) 22,500 IU LMWH/275 mg Sodium Caprate Delayed Release Tablet Manufacture

The above tablets were coated with a coating solution containing Eudragit L 12.5 (50%), isopropyl alcohol (44.45%), dibutyl sebecate (3%), talc (1.3%), and water (1.25%) in a Hi-Coater to a final % weight gain of 5.66%.

The resulting enteric coated tablets remained intact after 1 hour disintegration testing in pH 1.2 solution; complete disintegration was observed in pH 6.2 medium after 32-33 minutes.

### EXAMPLE 9

### Manufacture of Instant Release Capsule Dosage Form Containing LMWH and Enhancer

(a) 22,500 IU LMWH/275 mg Sodium Caprate Instant Release Capsule Manufacture

The granulate from the previous example, part a, was hand filled into Size 00 hard gelatin capsules to a target fill weight equivalent to the granulate content of the tablets in the previous example.

### EXAMPLE 10

### Manufacture of Delayed Release Tablet Dosage Form Containing LMWH without Enhancer

(a) LMWH Granulate Manufacture

A 500 g batch of parnaparin sodium: Avicel™ pH 101 (0.92:1) (FMC, Little Island, Co. Cork, Ireland) was granulated in a Gral 10 using a 50% aqueous solution of Kollidon 30 as the granulating solvent. The resulting granulate was dried for 60 minutes in a Niro Aeromatic Fluidized Bed Drier at an exhaust temperature of 38° C. The dried granulate was milled through a 30 mesh screen in a Fitzmill M5A. The potency of the resulting dried granulate was determined as 106.5% of the label claim.

(b) 22,500 IU LMWH Instant Release Tablet Manufacture

The above granulate (77.5%) was added to mannitol (21%) and Aerosil (1%) in a 25 L V coned blender and blended for 10 minutes. Magnesium stearate (0.5%) was added to the resulting blend and blending was continued for a further 1 minute. The resulting blend was tableted on Piccola tablet press using 13 mm round normal concave tooling to a mean tablet weight of 671 mg and a mean tablet hardness of 144 N.

The actual potency of the resulting tablets was determined as 21,651 IU LMWH per tablet.

(c) 22,500 IU LMWH Delayed Release Tablet Manufacture

The above tablets were coated with a coating solution containing Eudragit L 12.5 (50%), isopropyl alcohol (44.45%), dibutyl sebecate (3%), talc (1.3%), and water (1.25%) in a Hi-Coater to a final % weight gain of 4.26%.

The resulting enteric coated tablets remained intact after 1 hour disintegration testing in pH 1.2 solution; complete disintegration was observed in pH 6.2 medium in 22 minutes.

### EXAMPLE 11

### Effect of Controlled Release Dosage Form Containing Enhancer on the Systemic Availability of LMWH after Oral Administration in Dogs

(a) Dog Study Evaluation

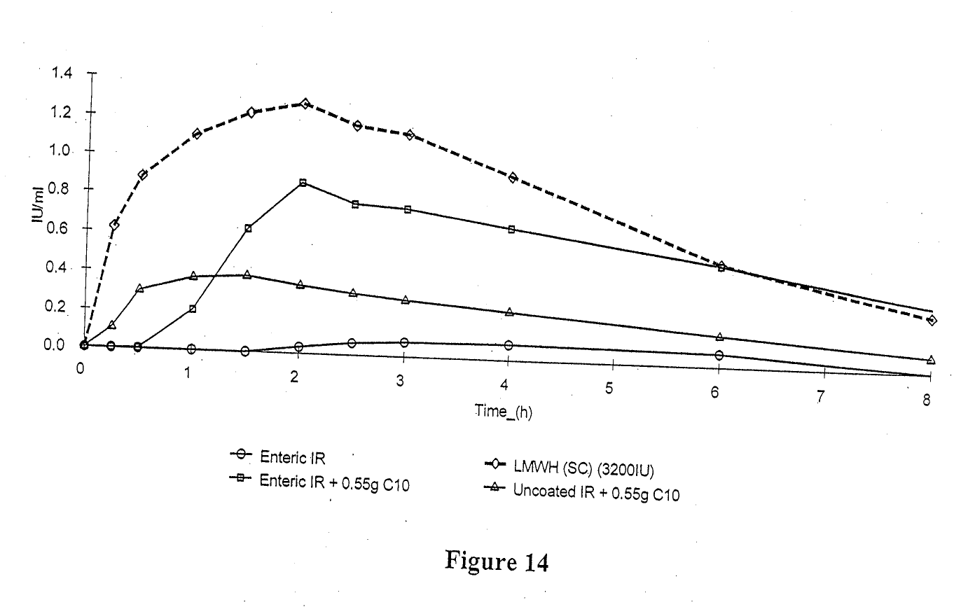

45,000 IU LMWH was administered to 8 beagle dogs (10.5-13.6 Kg), in an open label, non randomized crossed over block design, as (a) an instant release capsule dosage form containing 550 mg sodium caprate (equivalent to 2 capsules manufactured according to Example 9); (b) a delayed release tablet dosage containing 550 mg sodium caprate (equivalent to two tablets manufactured according to Example 8); and (c) a delayed release tablet dosage not containing any enhancer (equivalent to 2 tablets manufactured according to Example 10). 3,200 IU Fluxum™ SC was included in the study as a subcutaneous reference. Blood samples were taken from the jugular vein at various intervals and anti-factor Xₐ activity was determined. The resulting mean anti-factor Xₐ levels are shown in FIG. 14.

It should be noted that in the absence of sodium caprate, the systemic delivery of LMWH was minimal from the delayed release solid dosage form without enhancer. In contrast, a good anti-factor Xₐ response was observed after administration of the delayed release LMWH solid dosage form containing sodium caprate. The mean anti-factor Xₐ response from the delayed release dosage form containing sodium caprate was considerably higher than that from the instant release dosage form containing the same level of drug and enhancer.

### EXAMPLE 12

### Effect of the Site of Administration on the Systemic Availability of LMWH in Dogs after Co-administration with Enhancer

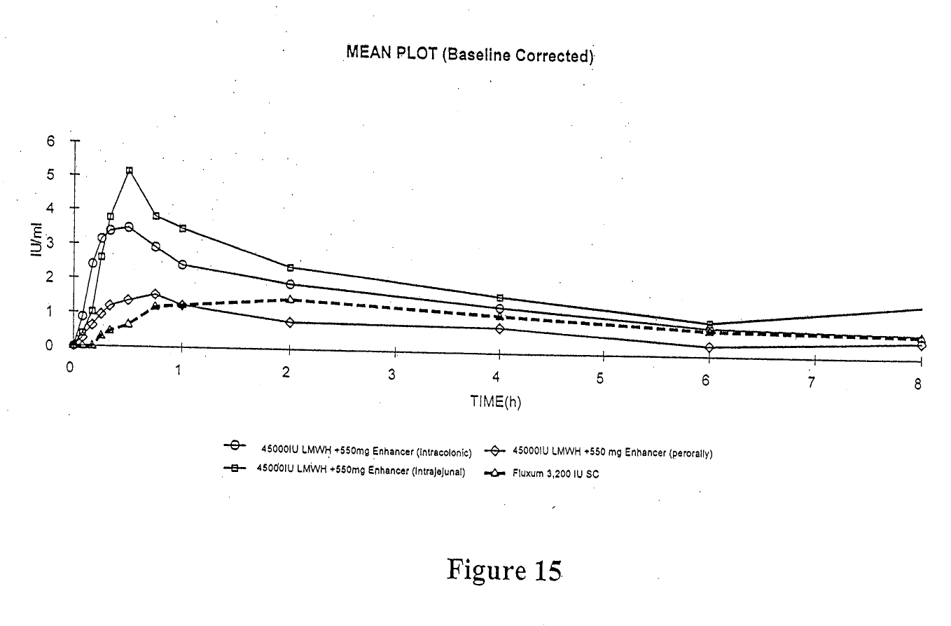

Four beagle dogs (10-15 Kg) were surgically fitted with catheters to the jejunum and colon respectively. The test solutions (10 ml) comprising LMWH with sodium caprate reconstituted in deionized water were administered to the dogs either orally or via the intra-intestinal catheters. 3,200 IU Fluxum™ SC was included in the study as a subcutaneous reference. Blood samples were taken from the brachial vein at various intervals and anti-factor Xₐ activity was determined. The resulting mean anti-factor Xₐ levels are shown in FIG. 15. The results show that the intestinal absorption of LMWH in the presence of enhancer is considerably higher than absorption from the stomach.

### EXAMPLE 13

### Leuprolide Containing Tablets

Following the same type of approach as used in Examples 1 and 2, leuprolide-containing IR tablets may be prepared according to the formulations detailed in Table 10.

### EXAMPLE 14

### A Bioequivalence Study of Formulations of Romidepsin in Beagle Dogs

A bioequivalency study in beagle dogs was undertaken with three experimental formulations of romidepsin to test several oral dosage forms of romidepsin and sodium caprate. The study was a single dose crossover study using from 2 to 5 dogs. Fasted animals were dosed weekly with an intravenous dose (reference) or one of three experimental romidepsin formulations administered directly into the duodenum via a surgically implanted cannula. In all cases the administered dose was 0.1 mg/kg body weight. Blood samples were obtained at selected time intervals post dosing and plasma was shipped to Japan Clinical Laboratories (JCL) for romidepsin analyses.

Upon receipt of bioanalytical data from JCL, the individual animal plasma data were loaded into an Excel spreadsheet (Microsoft® Office Excel 2003) and the following pharmacokinetic parameters were calculated from the concentration-time data for each subject: Cₘₐₓ, Tₘₐₓ, T_{1/2}, AUC₍₀₋ₜ₎ and % Bioavailability (% F). Pharmacokinetic parameters were calculated using macros written for Excel (Usansky et al., PK Functions for Microsoft Excel (1999) available at: www.boomer.org/pkin/xcel/pkf/pkf.doc). Percent F was calculated for the enhancer formulations by assuming the AUC for the intravenous doses to be equal to 100%.

Summary pharmacokinetic data for the three formulations are presented in Table 11, and detailed pharmacokinetic data for each formulation are presented in Tables 12-14.

TABLE 11
Summary Pharmacokinetic Data

| | IV Reference | Formula 1 | Formula 2 | Formula 3 |
|---|---|---|---|---|
| | Concen- | Concen- | Concen- | Concen- |
| | tration | tration | tration | tration |
| | (ng/ml) | (ng/ml) | (ng/ml) | (ng/ml) |
| Mean Cₘₐₓ | 32.00 | 5.50 | 5.15 | 2.23 |
| Mean Tₘₐₓ | 0.25 | 0.25 | 0.35 | 0.25 |
| Mean T_{1/2} | 0.23 | 0.19 | 0.27 | 0.17 |
| Mean AUC₍₀₋ₜ₎ | 13.11 | 2.25 | 4.02 | .83 |
| F (%) | 100 | 15.74 | 28.49 | 7.43 |
| N | 5 | 4 | 5 | 2 |

TABLE 12
Pharmacokinetic Data - Formulation 1

| | Dog 1 | Dog 2 | Dog 4 | Dog 5 |
|---|---|---|---|---|
| | Concen- | Concen- | Concen- | Concen- |
| | tration | tration | tration | tration |
| Time (hr) | (ng/ml) | (ng/ml) | (ng/ml) | (ng/ml) |
| 0.00 | 0.00 | 0.00 | 0.00 | 0.00 |
| 0.25 | 4.38 | 7.85 | 6.33 | 3.42 |
| 0.50 | 1.46 | 1.96 | 2.29 | 0.65 |
| 1.00 | 0.00 | 0.67 | 0.84 | 0.00 |
| 2.00 | 0.00 | 0.00 | 0.00 | 0.00 |
| 4.00 | 0.00 | 0.00 | 0.00 | 0.00 |
| 6.00 | 0.00 | 0.00 | 0.00 | 0.00 |
| 8.00 | 0.00 | 0.00 | 0.00 | 0.00 |
| Cₘₐₓ | 4.38 | 7.85 | 6.33 | 3.42 |
| Tₘₐₓ | 0.25 | 0.25 | 0.25 | 0.25 |
| T_{1/2} | 0.16 | 0.22 | 0.27 | 0.10 |
| AUC₍₀₋ₜ₎ | 1.64 | 3.20 | 3.07 | 1.10 |
| F (%) | 11.28 | 18.68 | 19.77 | 13.22 |

TABLE 13
Pharmacokinetic Data - Formulation 2

| Time (hr) | Dog 1 | Dog 2 | Dog 3 | Dog 4 | Dog 5 |
|---|---|---|---|---|---|
| 0.00 | 0.00 | 0.00 | 0.00 | 0.00 | 0.00 |
| 0.25 | 5.89 | 8.68 | 3.44 | 1.09 | 4.03 |
| 0.50 | 6.32 | 8.34 | 2.41 | 3.30 | 3.50 |
| 1.00 | 1.95 | 0.99 | 0.59 | 0.00 | 1.62 |
| 2.00 | 0.00 | 0.00 | 0.00 | 0.00 | 1.00 |
| 4.00 | 0.00 | 0.00 | 0.00 | 0.00 | 0.00 |
| 6.00 | 0.00 | 0.00 | 0.00 | 0.00 | 0.00 |
| 8.00 | 0.00 | 0.00 | 0.00 | 0.00 | 0.00 |
| Cₘₐₓ | 6.32 | 8.68 | 3.44 | 3.30 | 4.03 |
| Tₘₐₓ | 0.50 | 0.25 | 0.25 | 0.50 | 0.25 |
| T_{1/2} | 0.43 | 0.22 | 0.29 | −0.16 | 0.55 |
| AUC₍₀₋ₜ₎ | 5.31 | 6.04 | 2.20 | 1.51 | 5.04 |
| F (%) | 36.43 | 35.29 | 14.18 | 18.17 | 38.39 |

TABLE 14
Pharmacokinetic Data - Formulation 3

| Time (hr) | Dog 1 | Dog 5 |
|---|---|---|
| 0.00 | 0.00 | 0.00 |
| 0.25 | 2.83 | 1.62 |
| 0.50 | 0.76 | 0.70 |
| 1.00 | 0.00 | 0.00 |
| 2.00 | 0.00 | 0.00 |
| 4.00 | 0.00 | 0.00 |
| 6.00 | 0.00 | 0.00 |
| 8.00 | 0.00 | 0.00 |
| Cₘₐₓ | 2.83 | 1.62 |
| Tₘₐₓ | 0.25 | 0.25 |
| T_{1/2} | 0.13 | 0.21 |
| AUC₍₀₋ₜ₎ | 0.99 | 0.67 |
| F (%) | 6.81 | 8.05 |

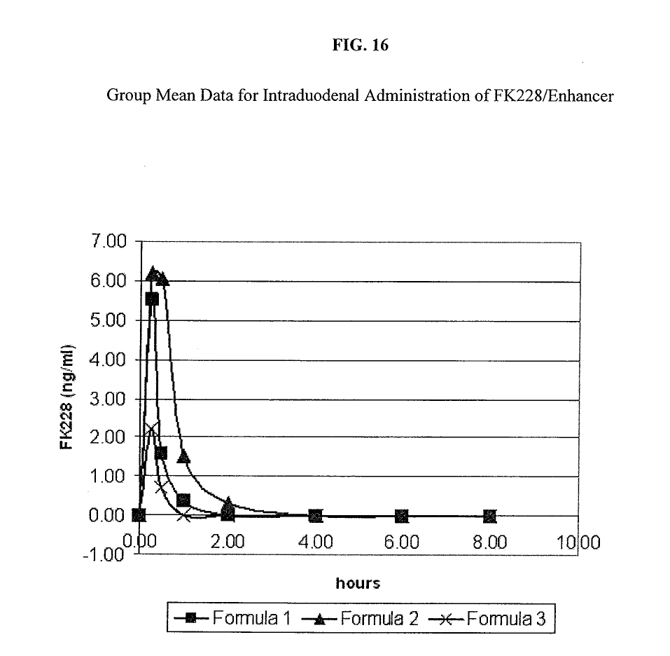

All animals received a romidepsin dose of 0.1 mg/kg, irrespective of route of administration, throughout the study. Bioavailability increased with increased amounts of sodium caprate in the formulae. Maximum oral bioavailability was observed with Formula 2, which contained the greatest amount of sodium caprate of any of the experimental formulations. The group mean data for the three intraduodenal dose groups are plotted in FIG. 16.

Solutions of romidepsin and sodium caprate administered to dogs by intraduodenal administration were bioavailable. Increasing concentrations of sodium caprate in the dosing solution resulted in increased absorption. The oral bioavailability of romidepsin was as high as 28% when given intraduodenally in solution with sodium caprate.

The compositions and dosage forms of the present invention also include the use of enhancers other than the medium chain fatty acids and medium chain fatty acid derivatives described above. Absorption enhancers such as fatty acids other than medium chain fatty acids; ionic, non-ionic and lipophilic surfactants; fatty alcohols; bile salts and bile acids; micelles; chelators and the like may be used to increase the bioavailability.

Nonionic surfactants considered within the scope of the invention include alkylglucosides; alkylmaltosides; alkylthioglucosides; lauryl macrogolglycerides; polyoxyalkylene ethers; polyoxyalkylene alkyl ethers; polyoxyalkylene alkylphenols; polyoxyalkylene alkyl phenol fatty acid esters; polyethylene glycol glycerol fatty acid esters; polyglycerol fatty acid esters; polyoxyalkylene sorbitan fatty acid esters; sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbates including polysorbate 20, polysorbate 40, polysorbate 60, polysorbate 65, polysorbate 80, polysorbate 85, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, sucrose monostearate, sucrose monolaurate, sucrose monopalmitate, PEG 10-100 nonyl phenol series, PEG 15-100 octyl phenol series, and poloxamers.

Ionic surfactants considered within the scope of the invention include alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; sodium laurylsulfate; and quaternary ammonium compounds.

Lipophilic surfactants considered within the scope of the invention include fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

Bile salts and acids considered within the scope of the invention include dihydroxy bile salts such as sodium deoxycholate, trihydroxy bile salts such as sodium cholate, cholic acid, deoxycholic acid, lithocholic acid, chenodeoxycholic acid (also referred to as “chenodiol” or “chenic acid”), ursodeoxycholic acid, taurocholic acid, taurodeoxycholic acid, taurolithocholic acid, taurochenodeoxycholic acid, tauroursodeoxycholic acid, glycocholic acid, glycodeoxycholic acid, glycolithocholic acid, glycochenodeoxycholic acid, and glycoursodeoxycholic acid.

Solubilizers considered within the scope of the invention include alcohols and polyols such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, mono-, di- and trgycerides of medium chain fatty acids and derivatives thereof; glycerides cyclodextrins and cyclodextrin derivatives; ethers of polyethylene glycols having an average molecular weight of about 200 to about 6000, such as tetrahydrofurfuryl alcohol PEG ether or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon.-caprolactone and isomers thereof, .delta.-valerolactone and isomers thereof, beta.-butyrolactone and isomers thereof; and other solubilizers known in the art, such as dimethyl acetamide, dimethyl isosorbide, N-methylpyrrolidones, monooctanoin, diethylene glycol monoethyl ether, and water.

Still other suitable surfactants will be apparent to those skilled in the art, and/or are described in the pertinent texts and literature.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A pharmaceutical composition which is effective in delivering therapeutically effective amounts of a drug and an enhancer, each as defined below, to an intestine, said composition comprising a DAC inhibitor and an enhancer wherein the enhancer comprises a medium chain fatty acid or a medium chain fatty acid derivative having a carbon chain length of from 6 to 20 carbon atoms and is solid at room temperature.

2. The composition of claim 1, wherein the carbon chain length is from 8 to 14 carbon atoms.

3. The composition of claim 1, wherein the carbon chain length is from 8 to 12 carbon atoms.

4. The composition of claim 1, wherein the carbon chain length is 8, 10, or 12 carbon atoms.

5. The composition of claim 1 wherein the enhancer is a sodium salt of a medium chain fatty acid.

6. The composition of claim 5, wherein the enhancer is selected from the group consisting of sodium caprylate, sodium caprate, and sodium laurate.

7. The composition of claim 1, wherein the drug and the enhancer are present in a ratio of from 1:100,000 to 10:1 (drug:enhancer).

8. The composition of claim 1 further comprising at least one auxiliary excipient.

9. The composition of claim 1, wherein the DAC inhibitor is selected from the group consisting of short-chain fatty acids, hydroxamic acids, propenamides, aroyl pyrrolyl hydroxyamides, trichostatins, spiruchostatins, salinamides, cyclic tetrapeptides, antanapeptins, cyclic-hydroxamic-acid-containing peptides, trapoxins, benzamides, tricyclic lactam derivatives, tricyclic sultam derivatives, acetate derivatives of amijiol, organosulfur compounds, psammaplins, and electrophilic ketones.

10. The composition of claim 1, wherein the DAC inhibitor is selected from the group consisting of butyrate, phenylbutyrate, pivaloyloxymethyl butyrate, N-Hydroxy-4-(3-methyl-2-phenyl-butyrylamino)-benzamide, 4-(2,2-Dimethyl-4-phenylbutyrylamino)-N-hydroxybenzamide, valproate, valproic acid, suberoyl-anilide hydroxamic acid, oxamflatin, M-carboxycinnamic acid bishydroxamide, 6-(3-benzoyl-ureido)-hexanoic acid hydroxyamide, suberic bishydroxamate, N-hydroxy-7-(2-naphthylthio) heptanomide, nicotinamide, scriptaid, scriptide, splitomicin, lunacin, ITF2357, A-161906, NVP-LAQ824, LBH589, pyroxamide, CBHA, 3-Cl-UCHA, SB-623, SB-624, SB-639, SK-7041, 3-(4-dimethylamino-phenyl)-N-hydroxy-2-propenamide, 2-amino-8-oxo-9,10-epoxy-decanoyl, 3-(4-aroyl-1H-pyrrol-2-yl)-N-hydroxy-2-propenamide, MC 1293, APHA Compound 8, trichostatin A, trichostatin C, trapoxin A, trapoxin B, romidepsin, HC-toxin, chlamydocin, antanapeptin A, antanapeptin B, antanapeptin C, antanapeptin D, diheteropeptin, WF-3161, Cyl-1, Cyl-2, apicidin, FR225497, FR901375, spiruchostatin A, spiruchostatin B, spiruchostatin C, salinamide A, salinamide B, M344, MS-275, CI-994, tacedinaline, sirtinol, diallyl disulfide, sulforaphane, α-ketoamide, trifluoromethylketone, pimeloylanilide o-aminoanilide, depudecin, psammaplin A, psammaplin F, tubacin, curcumin, histacin, pimeloylanilide o-aminoanilide, 6-Chloro-2,3,4,9-tetrahydro-1H-carbazole-1-carboxamide, CRA-024781, CRA-026440, CG1521, PXD101, G2M-777, CAY10398, CTPB, MGCD0103, and BL1521.

11. The composition of claim 1, wherein the DAC inhibitor is romidepsin.

12. A pharmaceutical composition which is effective in delivering therapeutically effective amounts of a romidepsin, said composition comprising:
romidepsin and an enhancer, wherein the enhancer is selected from the group consisting of sodium caprylate, sodium caprate, sodium laurate, and combination thereof.

13. The pharmaceutical composition of claim 12 further comprising at least one auxiliary excipient.

14. The pharmaceutical composition of claim 9b, wherein the auxiliary excipient is polyvinylpyrrolidone.

15. A solid oral dosage form comprising the composition of claim 1.

16. The dosage form of claim 15, wherein the dosage form is a tablet, a capsule, or a multiparticulate dosage form.

17. The dosage form of claim 15, wherein the dosage form is a delayed release dosage form.

18. The dosage form of claim 15, wherein the dosage form is a tablet.

19. The dosage form of claim 18, wherein the tablet is a multilayer tablet.

20. The dosage form of claim 15, wherein the dosage form further comprises a rate-controlling polymer material.

21. The dosage form of claim 20, wherein the rate-controlling polymer material is HPMC.

22. The dosage form of claim 20, wherein the rate-controlling polymer material is a polymer derived from acrylic or methacrylic acid and their respective esters or copolymers derived from acrylic or methacrylic acid and their respective esters.

23. The dosage form of claim 20, wherein the rate-controlling polymer material is a coating over the dosage form.

24. The dosage form of claim 23, wherein the tablet is a multilayer tablet.

25. The dosage form of claim 15, wherein the dosage form is a multiparticulate dosage form.

26. The dosage form of claim 25, wherein the multiparticulate dosage form comprises discrete particles, pellets, minitablets, or combinations thereof.

27. The dosage form of claim 26, wherein the multiparticulate dosage form comprises a blend of two or more populations of particles, pellets, minitablets, or combinations thereof each population having different in vitro and/or in vivo release characteristics.

28. The dosage form of claim 25, wherein the multiparticulate material is encapsulated in a gelatin capsule.

29. The dosage form of claim 28, wherein the capsule is coated with a rate-controlling polymer material.

30. The dosage form of claim 25, wherein the multiparticulate is incorporated into a sachet.

31. The dosage form of claim 26, wherein the discrete particles, pellets, minitablets, or combinations thereof are compressed into a tablet.

32. The dosage form of claim 31, wherein the tablet is coated with a rate controlling polymer material.

33. The dosage form of claim 31, wherein the tablet is a multilayer tablet.

34. The dosage form of claim 32, wherein the tablet is a multilayer tablet.

35. The dosage form of claim 15, wherein the DAC inhibitor and the enhancer are present in the dosage form in a ratio of from 1:100,000 to 10:1 (drug:enhancer).

36. The dosage form of claim 30, wherein the ratio is from 1:1,000 to 10:1 (drug:enhancer).

37. The dosage form of claim 10, wherein the DAC inhibitor is selected from the group consisting of short-chain fatty acids, hydroxamic acids, propenamides, aroyl pyrrolyl hydroxyamides, trichostatins, spiruchostatins, salinamides, cyclic tetrapeptides, antanapeptins, cyclic-hydroxamic-acid-containing peptides, trapoxins, benzamides, tricyclic lactam derivatives, tricyclic sultam derivatives, acetate derivatives of amijiol, organosulfur compounds, psammaplins, and electrophilic ketones.

38. The dosage form of claim 15, wherein the DAC inhibitor is selected from the group consisting of butyrate, phenylbutyrate, pivaloyloxymethyl butyrate, N-Hydroxy-4-(3-methyl-2-phenyl-butyrylamino)-benzamide, 4-(2,2-Dimethyl-4-phenylbutyrylamino)-N-hydroxybenzamide, valproate, valproic acid, suberoyl-anilide hydroxamic acid, oxamflatin, M-carboxycinnamic acid bishydroxamide, 6-(3-benzoyl-ureido)-hexanoic acid hydroxyamide, suberic bishydroxamate, N-hydroxy-7-(2-naphthylthio) heptanomide, nicotinamide, scriptaid, scriptide, splitomicin, lunacin, ITF2357, A-161906, NVP-LAQ824, LBH589, pyroxamide, CBHA, 3-Cl-UCHA, SB-623, SB-624, SB-639, SK-7041, 3-(4-dimethylamino-phenyl)-N-hydroxy-2-propenamide, 2-amino-8-oxo-9,10-epoxy-decanoyl, 3-(4-aroyl-1H-pyrrol-2-yl)-N-hydroxy-2-propenamide, MC 1293, APHA Compound 8, trichostatin A, trichostatin C, trapoxin A, trapoxin B, romidepsin, HC-toxin, chlamydocin, antanapeptin A, antanapeptin B, antanapeptin C, antanapeptin D, diheteropeptin, WF-3161, Cyl-1, Cyl-2, apicidin, FR225497, FR901375, spiruchostatin A, spiruchostatin B, spiruchostatin C, salinamide A, salinamide B, M344, MS-275, CI-994, tacedinaline, sirtinol, diallyl disulfide, sulforaphane, α-ketoamide, trifluoromethylketone, pimeloylanilide o-aminoanilide, depudecin, psammaplin A, psammaplin F, tubacin, curcumin, histacin, pimeloylanilide o-aminoanilide, 6-Chloro-2,3,4,9-tetrahydro-1H-carbazole-1-carboxamide, CRA-024781, CRA-026440, CG1521, PXD101, G2M-777, CAY10398, CTPB, MGCD0103, and BL1521.

39. The dosage form of claim 15, wherein the DAC inhibitor is romidepsin.

40. The dosage form of claim 15, comprising about 1 mg/m² to about 300 mg/m² of romidepsin.

41. The dosage form of claim 15, wherein the composition is in the form of a delayed release enteric coated tablet.

42. The dosage form of claim 41, wherein the DAC inhibitor and the enhancer are present in the dosage form in a ratio of from 1:1,000 to 10:1 (drug:enhancer).

43. The dosage form of claim 41, wherein the enhancer is sodium caprate.

44. The solid oral dosage form of claim 41, wherein the enhancer is sodium caprylate.

45. The solid dosage form of claim 41, wherein the enhancer is sodium laurate.

46. A pharmaceutical composition which is effective in delivering therapeutically effective amounts of an DAC inhibitor and an enhancer to an intestine, said composition comprising an DAC inhibitor and an enhancer, wherein the enhancer comprises:
(i) a salt of a medium chain fatty acid or salt thereof having a carbon chain length of from 6 to 20 carbon atoms;
(ii) a medium chain fatty acid halide derivative, a medium chain fatty acid anhydride derivative, or a medium chain fatty acid glyceride derivative, each of said derivatives having a carbon chain length of from 6 to 20 carbon atoms;
(iii) the fatty acid salt of clause (i) having, at the end opposite the fatty acid salt, an acid halide, acid anhydride, or glyceride moiety;
(iv) an acid halide derivative of clause (ii) above having, at the end opposite of the halide portion, an acid halide, acid anhydride, or glyceride moiety;
(v) an anhydride derivative of clause (ii) above having, at the end opposite of the anhydride, an acid anhydride, acid halide, or glyceride moiety; or
(vi) a glyceride derivative of clause (ii) above having, at the end opposite of the glyceride portion, a glyceride, acid halide, or acid anhydride moiety;
and wherein the enhancer is solid at room temperature.

47. A pharmaceutical composition which is effective in delivering therapeutically effective amounts of an DAC inhibitor and an enhancer to an intestine, said composition comprising an DAC inhibitor and an enhancer, wherein the enhancer: (1) comprises a medium chain fatty acid or a medium chain fatty acid derivative having a carbon chain length of from 6 to 20 carbon atoms; (2) is the only enhancer present in the composition; and (3) enhances intestinal delivery of the HDAC inhibitor to the underlying circulation.

48. The composition of claim 47, wherein the enhancer is a salt of a fatty acid having a carbon chain length of from 8 to 14 carbon atoms.

49. The composition of claim 47, wherein the carbon chain length is from 8 to 12 carbon atoms.

50. The composition of claim 47, wherein the carbon chain length is 8, 10, or 12 carbon atoms.

51. The composition of claim 50, wherein said fatty acid salt is a sodium salt.

52. The composition of claim 51, wherein said fatty acid salt is selected from the group consisting of sodium caprylate, sodium caprate, and sodium laurate.

53. The composition of claim 47, wherein the DAC inhibitor is selected from the group consisting of short-chain fatty acids, hydroxamic acids, propenamides, aroyl pyrrolyl hydroxyamides, trichostatins, spiruchostatins, salinamides, cyclic tetrapeptides, antanapeptins, cyclic-hydroxamic-acid-containing peptides, trapoxins, benzamides, tricyclic lactam derivatives, tricyclic sultam derivatives, acetate derivatives of amijiol, organosulfur compounds, psammaplins, and electrophilic ketones.

54. The composition of claim 47, wherein the DAC inhibitor is selected from the group consisting of butyrate, phenylbutyrate, pivaloyloxymethyl butyrate, N-Hydroxy-4-(3-methyl-2-phenyl-butyrylamino)-benzamide, 4-(2,2-Dimethyl-4-phenylbutyrylamino)-N-hydroxybenzamide, valproate, valproic acid, suberoylanilide hydroxamic acid, oxamflatin, M-carboxycinnamic acid bishydroxamide, 6-(3-benzoyl-ureido)-hexanoic acid hydroxyamide, suberic bishydroxamate, N-hydroxy-7-(2-naphthylthio) heptanomide, nicotinamide, scriptaid, scriptide, splitomicin, lunacin, ITF2357, A-161906, NVP-LAQ824, LBH589, pyroxamide, CBHA, 3-Cl-UCHA, SB-623, SB-624, SB-639, SK-7041, 3-(4-dimethylamino-phenyl)-N-hydroxy-2-propenamide, 2-amino-8-oxo-9,10-epoxy-decanoyl, 3-(4-aroyl-1H-pyrrol-2-yl)-N-hydroxy-2-propenamide, MC 1293, APHA Compound 8, trichostatin A, trichostatin C, trapoxin A, trapoxin B, romidepsin, HC-toxin, chlamydocin, antanapeptin A, antanapeptin B, antanapeptin C, antanapeptin D, diheteropeptin, WF-3161, Cyl-1, Cyl-2, apicidin, FR225497, FR901375, spiruchostatin A, spiruchostatin B, spiruchostatin C, salinamide A, salinamide B, M344, MS-275, CI-994, tacedinaline, sirtinol, diallyl disulfide, sulforaphane, α-ketoamide, trifluoromethylketone, pimeloylanilide o-aminoanilide, depudecin, psammaplin A, psammaplin F, tubacin, curcumin, histacin, pimeloylanilide o-aminoanilide, 6-Chloro-2,3,4,9-tetrahydro-1H-carbazole-1-carboxamide, CRA-024781, CRA-026440, CG1521, PXD101, G2M-777, CAY10398, CTPB, MGCD0103, and BL1521.

55. The composition of claim 47, wherein the DAC inhibitor is romidepsin.

56. The composition of claim 47, wherein the composition is in the form of a tablet, a capsule, or a multiparticulate composition.

57. The composition of claim 47, wherein the enhancer is selected from the group consisting of:
(a) an acid salt, acid halide, acid anhydride, or glyceride of a fatty acid having a carbon chain length of from 6 to 20 carbon atoms; and
(b) a derivative of clause (a) which is a difunctional in that it has on the end of the carbon chain opposite the acid salt group an acid halide, an acid anhydride, or a glyceride moiety.

58. The composition of claim 47, wherein the composition is solid at room temperature.

59. A process for the manufacture of a dosage form comprising the steps of:
a) providing a blend comprising an DAC inhibitor and an enhancer which is solid at room temperature and enhances intestinal delivery of the DAC inhibitor to the underlying circulation, wherein the enhancer comprises:
(i) a salt of a medium chain fatty acid having a carbon chain length of from 6 to 20 carbon atoms;
(ii) a medium chain fatty acid halide derivative, a medium chain fatty acid anhydride derivative, or a medium chain fatty acid glyceride derivative, each of said derivatives having a carbon chain length of from 6 to 20 carbon atoms;
(iii) the fatty acid salt of clause (i) having, at the end opposite the fatty acid salt, an acid halide, an acid anhydride, or glyceride moiety;
(iv) an acid halide derivative of clause (ii) above having, at the end opposite of the halide portion, an acid halide, acid anhydride, or glyceride moiety;
(v) an anhydride derivative of clause (ii) above having, at the end opposite of the anhydride, an acid anhydride, acid halide, or glyceride moiety; or
(vi) a glyceride derivative of clause (ii) above having, at the end opposite of the glyceride portion, a glyceride, an acid halide, or acid anhydride moiety; and
b) forming the solid oral dosage form from the blend.

60. The process of claim 59, wherein the forming step comprises direct compression of the blend into a tablet.

61. The process of claim 59, wherein the forming step comprises granulating the blend to form granules for incorporation into said solid oral dosage form.

62. The process of claim 59, wherein the forming step comprises encapsulating the blend.

63. The process of claim 59 further comprising the step of forming an enteric coating on the solid oral dosage form.

64. The process of claim 59, wherein the DAC inhibitor is selected from the group consisting of short-chain fatty acids, hydroxamic acids, propenamides, aroyl pyrrolyl hydroxyamides, trichostatins, spiruchostatins, salinamides, cyclic tetrapeptides, antanapeptins, cyclic-hydroxamic-acid-containing peptides, trapoxins, benzamides, tricyclic lactam derivatives, tricyclic sultam derivatives, acetate derivatives of amijiol, organosulfur compounds, psammaplins, and electrophilic ketones.

65. The process of claim 59, wherein the DAC inhibitor is selected from the group consisting of butyrate, phenylbutyrate, pivaloyloxymethyl butyrate, N-Hydroxy-4-(3-methyl-2-phenyl-butyrylamino)-benzamide, 4-(2,2-Dimethyl-4-phenylbutyrylamino)-N-hydroxybenzamide, valproate, valproic acid, suberoyl-anilide hydroxamic acid, oxamflatin, M-carboxycinnamic acid bishydroxamide, 6-(3-benzoyl-ureido)-hexanoic acid hydroxyamide, suberic bishydroxamate, N-hydroxy-7-(2-naphthylthio) heptanomide, nicotinamide, scriptaid, scriptide, splitomicin, lunacin, ITF2357, A-161906, NVP-LAQ824, LBH589, pyroxamide, CBHA, 3-Cl-UCHA, SB-623, SB-624, SB-639, SK-7041, 3-(4-dimethylamino-phenyl)-N-hydroxy-2-propenamide, 2-amino-8-oxo-9,10-epoxy-decanoyl, 3-(4-aroyl-1H-pyrrol-2-yl)-N-hydroxy-2-propenamide, MC 1293, APHA Compound 8, trichostatin A, trichostatin C, trapoxin A, trapoxin B, romidepsin, HC-toxin, chlamydocin, antanapeptin A, antanapeptin B, antanapeptin C, antanapeptin D, diheteropeptin, WF-3161, Cyl-1, Cyl-2, apicidin, FR225497, FR901375, spiruchostatin A, spiruchostatin B, spiruchostatin C, salinamide A, salinamide B, M344, MS-275, CI-994, tacedinaline, sirtinol, diallyl disulfide, sulforaphane, α-ketoamide, trifluoromethylketone, pimeloylanilide o-aminoanilide, depudecin, psammaplin A, psammaplin F, tubacin, curcumin, histacin, pimeloylanilide o-aminoanilide, 6-Chloro-2,3,4,9-tetrahydro-1H-carbazole-1-carboxamide, CRA-024781, CRA-026440, CG1521, PXD101, G2M-777, CAY10398, CTPB, MGCD0103, and BL1521.

66. The process of claim 59, wherein the DAC inhibitor is romidepsin.

67. A method for the treatment or prevention of a medical condition comprising the step of administering orally to a patient a therapeutically effective amount of the composition of claim 1.

68. The method of claim 67, wherein the medical condition is cancer.

69. The method of claim 67, wherein the medical condition is a proliferative disease.

70. The method of claim 67, wherein the medical condition is an anti-inflammatory disease.

71. The method of claim 67, wherein the medical condition is an autoimmune disease.

72. The method of claim 71, wherein the DAC inhibitor is selected from the group consisting of short-chain fatty acids, hydroxamic acids, propenamides, aroyl pyrrolyl hydroxyamides, trichostatins, spiruchostatins, salinamides, cyclic tetrapeptides, antanapeptins, cyclic-hydroxamic-acid-containing peptides, trapoxins, benzamides, tricyclic lactam derivatives, tricyclic sultam derivatives, acetate derivatives of amijiol, organosulfur compounds, psammaplins, and electrophilic ketones.

73. The method of claim 71, wherein the DAC inhibitor is selected from the group consisting of butyrate, phenylbutyrate, pivaloyloxymethyl butyrate, N-Hydroxy-4-(3-methyl-2-phenyl-butyrylamino)-benzamide, 4-(2,2-Dimethyl-4-phenylbutyrylamino)-N-hydroxybenzamide, valproate, valproic acid, suberoyl-anilide hydroxamic acid, oxamflatin, M-carboxycinnamic acid bishydroxamide, 6-(3-benzoyl-ureido)-hexanoic acid hydroxyamide, suberic bishydroxamate, N-hydroxy-7-(2-naphthylthio) heptanomide, nicotinamide, scriptaid, scriptide, splitomicin, lunacin, ITF2357, A-161906, NVP-LAQ824, LBH589, pyroxamide, CBHA, 3-Cl-UCHA, SB-623, SB-624, SB-639, SK-7041, 3-(4-dimethylamino-phenyl)-N-hydroxy-2-propenamide, 2-amino-8-oxo-9,10-epoxy-decanoyl, 3-(4-aroyl-1H-pyrrol-2-yl)-N-hydroxy-2-propenamide, MC 1293, APHA Compound 8, trichostatin A, trichostatin C, trapoxin A, trapoxin B, romidepsin, HC-toxin, chlamydocin, antanapeptin A, antanapeptin B, antanapeptin C, antanapeptin D, diheteropeptin, WF-3161, Cyl-1, Cyl-2, apicidin, FR225497, FR901375, spiruchostatin A, spiruchostatin B, spiruchostatin C, salinamide A, salinamide B, M344, MS-275, CI-994, tacedinaline, sirtinol, diallyl disulfide, sulforaphane, α-ketoamide, trifluoromethylketone, pimeloylanilide o-aminoanilide, depudecin, psammaplin A, psammaplin F, tubacin, curcumin, histacin, pimeloylanilide o-aminoanilide, 6-Chloro-2,3,4,9-tetrahydro-1H-carbazole-1-carboxamide, CRA-024781, CRA-026440, CG 1521, PXD101, G2M-777, CAY10398, CTPB, MGCD0103, and BL1521.

74. The method of claim 71, wherein the DAC inhibitor is romidepsin.

75. A method for the treatment or prevention of a medical condition comprising the step of administering orally to a patient a therapeutically effective amount of the composition of claim 47.

76. The method of claim 75, wherein the medical condition is cancer.

77. The method of claim 75, wherein the medical condition is a proliferative disease.

78. The method of claim 75, wherein the medical condition is an anti-inflammatory disease.

79. The method of claim 75, wherein the medical condition is an autoimmune disease.

80. The method of claim 75, wherein the DAC inhibitor is selected from the group consisting of short-chain fatty acids, hydroxamic acids, propenamides, aroyl pyrrolyl hydroxyamides, trichostatins, spiruchostatins, salinamides, cyclic tetrapeptides, antanapeptins, cyclic-hydroxamic-acid-containing peptides, trapoxins, benzamides, tricyclic lactam derivatives, tricyclic sultam derivatives, acetate derivatives of amijiol, organosulfur compounds, psammaplins, and electrophilic ketones.

81. The method of claim 75, wherein the DAC inhibitor is selected from the group consisting of butyrate, phenylbutyrate, pivaloyloxymethyl butyrate, N-Hydroxy-4-(3-methyl-2-phenyl-butyrylamino)-benzamide, 4-(2,2-Dimethyl-4-phenylbutyrylamino)-N-hydroxybenzamide, valproate, valproic acid, suberoyl-anilide hydroxamic acid, oxamflatin, M-carboxycinnamic acid bishydroxamide, 6-(3-benzoyl-ureido)-hexanoic acid hydroxyamide, suberic bishydroxamate, N-hydroxy-7-(2-naphthylthio) heptanomide, nicotinamide, scriptaid, scriptide, splitomicin, lunacin, ITF2357, A-161906, NVP-LAQ824, LBH589, pyroxamide, CBHA, 3-Cl-UCHA, SB-623, SB-624, SB-639, SK-7041, 3-(4-dimethylamino-phenyl)-N-hydroxy-2-propenamide, 2-amino-8-oxo-9,10-epoxy-decanoyl, 3-(4-aroyl-1H-pyrrol-2-yl)-N-hydroxy-2-propenamide, MC 1293, APHA Compound 8, trichostatin A, trichostatin C, trapoxin A, trapoxin B, romidepsin, HC-toxin, chlamydocin, antanapeptin A, antanapeptin B, antanapeptin C, antanapeptin D, diheteropeptin, WF-3161, Cyl-1, Cyl-2, apicidin, FR225497, FR901375, spiruchostatin A, spiruchostatin B, spiruchostatin C, salinamide A, salinamide B, M344, MS-275, CI-994, tacedinaline, sirtinol, diallyl disulfide, sulforaphane, α-ketoamide, trifluoromethylketone, pimeloylanilide o-aminoanilide, depudecin, psammaplin A, psammaplin F, tubacin, curcumin, histacin, pimeloylanilide o-aminoanilide, 6-Chloro-2,3,4,9-tetrahydro-1H-carbazole-1-carboxamide, CRA-024781, CRA-026440, CG 1521, PXD101, G2M-777, CAY10398, CTPB, MGCD0103, and BL1521.

82. The method of claim 75, wherein the DAC inhibitor is romidepsin.
